(19) United States
(12) Patent Application Publication
(10) Pub. No.: US 2005/0266420 A1
(43) Pub. Date: Dec. 1, 2005

(54) Multigene predictors of response to chemotherapy

(75) Inventors: Lajos Pusztai, Pearland, TX (US); W. Fraser Symmans, Houston, TX (US); Kenneth R. Hess, Houston, TX (US); Mark Ayers, Pennington, NJ (US); James Stec, Plymouth, MA (US)

Correspondence Address: Charles P. Landrum;Fulbright & Jaworski L.L.P., Suite 2400, 600 Congress Avenue, Austin, TX, 78701, US

(73) Assignees: Board of Regents, The University of Texas System; Millenium Pharmaceuticals, Inc.

(21) Appl. No.: 10/955,054

(22) Filed: Sep. 30, 2004

Related U.S. Application Data
(60) Provisional application No. 60/575,308, filed on May 28, 2004.

(51) Int. Cl. C12Q 1/68; G06F 19/00; G01N 33/48; G01N 33/50

(52) U.S. Cl. 435/6; 702/19; 702/20

(57) ABSTRACT

The present invention provides the identification of genes that are expressed in tumors that are responsive to a given therapeutic agent and whose expression (either increased expression or decreased expression) correlates with responsiveness to that therapeutic agent. One or more of the genes of the present invention can be used as markers (or surrogate markers) to identify tumors that are likely to be successfully treated by that agent.

This application claims priority to U.S. Provisional Patent application Ser. No. 60/575,308, filed on May 28, 2004, entitled “Multigene Predictors of Response to Chemotherapy,” which is incorporated herein by reference in its entirety.

### BACKGROUND OF THE INVENTION

1. Field of the Invention

The present invention relates generally to the field of cancer biology. More particularly, it concerns gene expression profiles that are indicative of the responsiveness of a cancer to therapy. In specific embodiments, the invention concerns gene expression profiles in paclitaxel/5-fluorouracil (5-FU), doxorubicine, and cyclophosphamide (P/FAC)-sensitive and P/FAC-resistant cancer.

2. Description of Related Art

Cancers can be viewed as a breakdown in the communication between tumor cells and their environment, including their normal neighboring cells. Normally, cells do not divide in the absence of stimulatory signals or in the presence of inhibitory signals. In a cancerous or neoplastic state, a cell acquires the ability to “override” these signals and to proliferate under conditions in which a normal cell would not.

In general, tumor cells must acquire a number of distinct aberrant traits in order to proliferate in an abnormal manner. Reflecting this requirement is the fact that the genomes of certain well-studied tumors carry several different independently altered genes, including activated oncogenes and inactivated tumor suppressor genes. In addition to abnormal cell proliferation, cells must acquire several other traits for tumor progression to occur. For example, early on in tumor progression, cells must evade the host immune system. Further, as tumor mass increases, the tumor must acquire vasculature to supply nourishment and remove metabolic waste. Additionally, cells must acquire an ability to invade adjacent tissue. In many cases cells ultimately acquire the capacity to metastasize to distant sites.

It is apparent that the complex process of tumor development and growth must involve multiple gene products. It is therefore important to identify the genes and gene products that can serve as targets for the diagnosis, prevention and treatment of cancers. Historically, research has focused on exploring the prognostic or predictive value of individual molecules expressed by human cancers. The general approach has been to take a biologically important molecule and examine whether its presence or absence correlates with clinical outcome. Unfortunately, the association of putative markers with clinical outcome is often weak and is rarely independent of other clinical characteristics, which limits its usefulness in clinical decision making.

The limited utility of individual molecules to predict clinical outcome of cancer may be due to the incomplete understanding of the function of these markers. In addition, biologically important molecules act in concert and form complex, interactive pathways where an individual molecule may only contribute limited information on the functional activity of a whole pathway. The promise of microarray technology is that by assessing the transcriptional activity of a large number of genes, the complex gene-expression profile may contain more information than any individual molecule that contributes to it.

There are examples indicating that the molecular classification of cancer based on gene-expression profiles is possible. Unsupervised clustering of breast cancer specimens consistently separated tumors into ER⁺ and ER⁻ clusters (Perou et al., 2000; Pusztai et al., 2003; Gruvberger et al., 2001). Analysis of gene-expression profiles also distinguished sporadic breast cancers from breast cancer gene, BRCA, mutant cases (Hedenfalk et al., 2001).

Transcriptional profiles also revealed previously unrecognized molecular subgroups within existing histological categories in breast cancer (Perou et al., 2000), diffuse large-B-cell lymphoma, and soft tissue and central nervous system embryonal tumors (Nielsen et al., 2002; Pomeroy et al., 2002). In addition, gene-expression profiles have been shown to predict survival of patients with node-negative breast cancer (van't Veer et al., 2002; van de Vijver et al., 2002), lymphoma (Alizadeh et al., 2000; Rosenwald, 2002), renal cancer (Takahashi et al., 2001), and lung cancer (Beer et al., 2002).

Another possible clinical application of microarray technology is in predicting a patient's response to anti-cancer therapy. The number of anti-cancer drugs and multi-drug combinations has increased substantially in the past decade, however, treatments continue to be applied empirically using a trial-and-error approach. Clinical experience shows that some tumors are sensitive to several different types of chemotherapeutic agents, while other cancers of the same histology show selective sensitivity to certain drugs but resistance to others. A test that could assist physicians to select the optimal chemotherapy from several alternative treatment options would be an important clinical advance.

### SUMMARY OF THE INVENTION

Embodiments of the invention include methods for assessing the responsiveness of a tumor to therapy. In certain embodiments the methods comprise obtaining a sample of a tumor from a patient; evaluating the sample for expression of one or more markers identified in Table 1; and assessing the responsiveness of the tumor to therapy based on the evaluation of marker expression in the sample. Marker refers to a gene or gene product (RNA or polypeptide) whose expression is related to response of a cancer to a therapy, either a positive (complete pathological response) or a negative response (residual disease). Expression of a marker may be assessed by detecting polynucleotides or polypeptides derived therefrom. In particular emobodiments, the marker is the nucleic acid encoding the microtubule-associated protein Tau or the encoded Tau polypeptide. In certain aspects, the tumor may be classified as sensitive when the therapy achieves an outcome of a complete pathological response or the gene expression profiles predicts that a tumor will have some probability of a complete pathological response. In still further aspects of the invention, the chance of a complete pathological response in a patient's tumor may be 35, 40, 45, 50, 55, 60, 65, 70, 80, 90, 95% or any value therebetween. In other aspects, the tumor may be classified as resistant to therapy, when the therapy does not achieve an outcome of a significant pathological response or the gene expression profiles predicts that a tumor will have some probability that the response will not achieve a pathological response. In still further aspects of the invention, the chance of a complete pathological response in a resistant cell may be 30, 25, 20, 15, 10% or less, including any value therebetween.

In certain embodiments, the therapy is a chemotherapy, and preferably P/FAC therapy. In certain aspects of the invention, evaluating the expression (gene expression profile) of the one or more markers comprises using a prediction algorithm. In further embodiments, the algorithm is k-nearest neighbor, support vector machines, diagonal linear discriminant analyses, or compound co-variate predictor, preferably a k-nearest neighbor algorithm. In certain aspects, a k-nearest neighbor algorithm will have, for example, a k value of 1, 2, 3, 4, 5, 6, 7, 8, 9, 10, 11, 12, 13, 14, 15, 16, 17, 18, 19, or 20. In preferred embodiments k=7.

In certain aspects of the invention, the tumor comprises breast cancer. In still other aspects the tumor is sampled by aspiration, biopsy, or surgical resection. Embodiments of the invention include assessing the expression of the one or more markers by detecting a mRNA derived from one or more markers. In a preferred embodiment, detection comprises microarray analysis, and more preferably the microarray is an Affymetrix Gene Chip. In other aspects of the invention, detection comprises nucleic acid amplification, preferably PCR. In still further aspects, detection is by in situ hybridization. In further embodiments, assessing the expression of one or more markers is by detecting a protein derived from a gene identified as a marker. A protein may be detected by immunohistochemistry, western blotting, or other known protein detection means.

In still a further embodiment includes methods of monitoring a cancer patient receiving a chemotherapy, preferably P/FAC therapy. Methods of monitoring a cancer patient comprise obtaining a tumor sample from the patient during chemotherapy; evaluating expression of one or more markers of Table 1 in the tumor sample; and assessing the cancer patient's responsiveness to chemotherapy, e.g., P/FAC therapy. A tumor sample may be obtained, evaluated and assessed repeatedly at various time points during chemotherapy.

Accordingly, in certain aspects it would be useful to identify genes and/or gene products that represent prognostic genes with respect to the response to a given therapeutic agent or class of therapeutic agents. It then may be possible to determine which patients will benefit from particular therapeutic regimen and, importantly, determine when, if ever, the therapeutic regime begins to lose its effectiveness for a given patient. The ability to make such predictions would make it possible to discontinue a therapeutic regime that has lost its effectiveness well before its loss of effectiveness becomes apparent by conventional measures.

In yet other embodiments include methods of assessing anti-cancer activity of a candidate substance. The methods comprise contacting a first cancer cell with a candidate substance; comparing expression of one or more markers in Table 1 in a first cancer cell exposed to a candidate substance with expression of the markers in a second cancer cell not contacted with the candidate substance; and assessing the anti-cancer activity of the candidate substance. Anti-cancer activity can be the sensitization of a cancer cell to therapy, which may be evaluated by gene expression profiles. In certain aspects, the therapy is a chemotherapy, preferably the chemotherapy is P/FAC therapy. For example, the anticancer efficacy of trastuzumab may be assessed as well as its ability to increase the sensitivity of cancer to chemotherapy (U.S. Pat. Nos. 6,399,063; 6,387,371; 6,165,464; 5,772,997; and 5,677,171, each of which is incorporated herein by reference in its entirety).

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a dot plot of the fully cross-validated misclassification results for the DLDA classifier with 30 genes over the 100 iterations for 2-, 5-, 7-, 10-, 15-, 20-, 40- and 82-fold cross-validation.

FIG. 2 illustrates the Area Above the ROC curves (AAC) results for 2-fold CV plotting against the number of top genes included. Data for 14 classifier methods with different numbers of genes included (39 subset sizes) are shown (means over the 100 iterations). Horizontal dotted lines indicate the mean+/−2 SD for the DLDA classifier with 30 genes.

FIG. 3 illustrates Misclassification Error Rates (MER) for 2-fold CV plotted against the number of top genes included. Data for 14 classifiers and 39 gene subset sizes are shown (means over the 100 iterations). Horizontal lines are drawn at the mean+/−2 SD for DLDA with 30 genes.

FIG. 4 illustrates Area Above the ROC curves (AAC) results for 5-fold CV plotted against the number of top genes included. Data for 14 classifiers and 39 gene subset sizes are shown (means over the 100 iterations). Horizontal lines are drawn at the mean+/−2 SD for DLDA with 30 genes.

FIGS. 5A-5C. show microtubule associated protein Tau mRNA expression measured by Affymetrix U133A chip in 60 breast cancer patients. (FIG. 5A) The location of the target sequences for the 4 distinct Affymetrix probe sets is shown along the Tau cDNA (FIG. 5B) Heat map of Tau expression in each of the specimens. Each column represents a patient sample; each row represents a probe set. High and low expression are typically color coded in red and green, respectively. (FIG. 5C) Tau mRNA expression measured by each of the 4 probe sets is significantly lower in the cohort of patients with pathological CR compared to those with residual disease (Mann-Whitney test).

FIGS. 6A-6F. illustrated validation of Tau expression by immunohistochemistry on a tissue-array from an independent set of patients who received similar preoperative chemotherapy (n=122). FIG. 6A illustrates Tau protein expression in normal breast epithelial cells and blood vessels, (FIG. 6B) shows weak 1+, (FIG. 6C) moderate 2+, and (FIG. 6D) strong 3+ staining in invasive tumor cells (Magnification ×40). The patient represented in FIG. 6B achieved a pathologic CR whereas the patient with the tumor represented in FIG. 6D had extensive residual disease. The bar graphs (FIG. 6E) show the proportion of patients with pathological CR and residual disease among Tau-positive and Tau-negative cases, respectively (chi-square test). Forty-four % of Tau-negative patients had pathological CR compared to 17% of Tau-positive cases. (FIG. 6F) Multivariate analysis of predictive factors for pathological CR identified higher nuclear grade, younger age and Tau-negative status as significant independent predictors of pathological CR (logistic regression analysis).

FIGS. 7A-7D. illustrate the effect of Tau down regulation on the sensitivity of ZR75.1 breast cancer cells to paclitaxel and epirubicin. (FIG. 7A) Twelve breast cancer cell lines were screened for Tau expression by Western-Blot and 4 cell lines were positive. (FIG. 7B) Tau protein expression was down regulated in ZR75.1 cells by Tau siRNA transfection in a time dependent manner. (FIGS. 7C and 7D) Dose response curves of parental, lamin siRNA and Tau siRNA transfected ZR75.1 cells after 48 H exposure to paclitaxel or epirubicin. ATP assay results of triplicate experiments and 95% confidence intervals are plotted. Tau siRNA increases sensitivity to paclitaxel but not to epirubicin.

FIGS. 8A-8G. show fluorescent paclitaxel uptake by Tau knock down cells. FACS analysis of ZR75.1 cells transfected with lamin siRNA (FIG. 8A) and Tau siRNA (FIG. 8B), after exposure to Oregon green fluorescent paclitaxel. (FIG. 8C) Percentage of cells with >10 arbitrary fluorescent units at 20, 50 and 80 minutes after incubation with 1 μM fluorescent paclitaxel. Cells transfected with Tau siRNA show increased percentage of fluorescent cells compared to control or lamin siRNA transfected cells. FACS analysis of spontaneously fluorescent epirubicin uptake in lamin knocked-down (FIG. 8D) and Tau knocked-down cells (FIG. 8E). Fluorescent microscopy showing that fluorescent paclitaxel is located in the cytoplasm (FIG. 8F) and also binds to the mitotic spindle during anaphase (FIG. 8G) in cells with low Tau-expression.

FIGS. 9A-9C. illustrates that Tau partially protects tubulin from paclitaxel-induced polymerization in vitro. Effects of paclitaxel and Tau and the combination of the two on microtubule polymerization. Tubulin (20 μM) and GTP buffer were incubated at 37° C. alone (x) or with 20 μM paclitaxel (o), 15 μM microtubule associated protein Tau (▪), or 20 μM paclitaxel and 15 ∝M microtubule associated protein Tau (●) for 30 min. Polymerization is measured as increasing optical density (A340) at 30-second intervals. (FIG. 9A) Simultaneous exposure to paclitaxel and Tau augmented tubulin polymerisation. (FIG. 9B) Pre-incubation of tubulin with Tau decreased paclitaxel-induced microtubule polymerisation. Tubulin was incubated with 2 concentrations of Tau (15 μM or 7.5 μM) at 37° C. for 30 minutes before adding paclitaxel (20 μM). Tau decreased the paclitaxel-induced polymerisation in a dose-dependent manner. (FIG. 9C) Competition between Tau and paclitaxel binding to tubulin was assessed using fluorescent paclitaxel. Tubulin was incubated directly with 5 μM of fluorescent paclitaxel or it was pre-incubated with regular paclitaxel (20 μM) or microtubule associated protein Tau (15 μM) for 30 minutes before fluorescent paclitaxel was added. Tubulin-bound fluorescence was measured and indicated reduced fluorescence in the presence of regular paclitaxel or Tau. This demonstrates that preincubation with Tau reduces the ability of paclitaxel to bind to tubulin.

### DESCRIPTION OF ILLUSTRATIVE EMBODIMENTS

Currently, there are at least 4 commonly used pre- or post-operative chemotherapy regimens for stage I-III breast cancers. Prior to the present invention, there were few tests to select the best regimen for an individual prior to the start of chemotherapy. Typically, treatments were evaluated empirically using a trial-and-error approach. Complete pathologic eradication of breast cancer from the breast (and regional lymph nodes) predicts cure with high accuracy. However, this endpoint is only available after completion of the empirically selected chemotherapy. In the case of P/FAC chemotherapy, the course of treatment last 6 months, and only between 15-30% of the patients achieve a pathological complete response (pCR).

The ability to choose an appropriate treatment at the outset may make the difference between cure and recurrence of a cancer, such as breast cancer. The present invention provides for the identification of patients who are the most likely to benefit from a therapy, such as P/FAC chemotherapy, by assessing the differential expression of one or more of the responsiveness genes in a tumor sample from a patient. In one example, it is estimated that an individual will experience complete pathological response to P/FAC therapy with an estimated 66% positive predictive value. A predictive value as used herein is the percentage of patients predicted to have a certain therapeutic outcome that do actually have the predicted therapeutic outcome. A therapeutic outcome may range from cure to no benefit and may include the slowing of tumor growth, a reduction in tumor burden, eradication of the tumor as determined by pathology, and other therapeutic outcomes. This represents a doubling of the chance of achieving complete pathological response (and likely cure) from P/FAC chemotherapy from 15-30% in untested patients to 66% in patients who would be selected to receive P/FAC chemotherapy on the basis of the proposed test results, using this example of the inventive methods. For these patients a P/FAC regimen represents the best chance of cure over the unselected use of treatments. Such predictive test can be used to select patients for this treatment regimen either as pre- or postoperative treatment. These genes alone or in combination may also be used as therapeutic targets to develop novel drugs against breast cancer or to modulate and increase the activity of existing therapeutic agents.

The expression level of a set or subset of identified responsiveness gene(s), or the proteins encoded by the responsive genes, may be used to: 1) determine if a tumor can be or is likely to be successfully treated by an agent or combination of agents; 2) determine if a tumor is responding to treatment with an agent or combination of agents; 3) select an appropriate agent or combination of agents for treating a tumor; 4) monitor the effectiveness of an ongoing treatment; and 5) identify new treatments (either single agent or combination of agents). In particular, the identified responsiveness genes may be utilized as markers (surrogate and/or direct) to determine appropriate therapy, to monitor clinical therapy and human trials of a drug being tested for efficacy, and to develop new agents and therapeutic combinations.

In certain embodiments, methods and compositions include genes (markers) that are expressed in cancer cells responsive to a given therapeutic agent and whose expression (either increased expression or decreased expression) correlates with responsiveness to a therapeutic agent, see Table 1. A “responsiveness gene” or “gene marker” as used herein is a gene whose increased expression or decreased expression is correlated with a cell's response to a particular therapy. A response may be either a therapeutic response (sensitivity) or a lack of therapeutic response (residual disease, which may indicate resistance). Accordingly, one or more of the genes of the present invention can be used as markers (or surrogate markers) to identify tumors and tumor cells that are likely to be successfully treated by a therapeutic agent(s). In addition, the markers of the present invention can be used to identify cancers that have become or are at risk of becoming refractory to a treatment. Aspects of the invention include marker sets that can identify patients that are likely to respond or not to respond to a therapy.

In still further embodiments, the invention is directed to methods of treating or sensitizing a tumor in an individual to chemotherapy. These methods may comprise the steps of: administering to the individual an agent that reduces the level of a gene whose down regulation is associated with pCR, e.g., Tau; thus sensitizing the tumor to chemotherapeutic agent such as paclitaxel; and administering an effective amount of a chemotherapeutic agent, such as paclitaxel. This method would be generally used to treat tumors which are resistant to chemotherapy, including breast tumors, glioblastomas, medulloblastomas, pancreatic adenocarcinomas, lung carcinomas, melanomas, and the like.

As used herein, cancer cells, including tumor cells, are “responsive” to a therapeutic agent if its rate of growth is inhibited or the tumor cells die as a result of contact with the therapeutic agent, compared to its growth in the absence of contact with the therapeutic agent. The quality of being responsive to a therapeutic agent is a variable one, with different tumors exhibiting different levels of “responsiveness” to a given therapeutic agent, under different conditions. In one embodiment of the invention, tumors may be predisposed to responsiveness to an agent if one or more of the corresponding responsiveness markers are expressed.

Cancer, including tumor cells, are “non-responsive” to a therapeutic agent if its rate of growth is not inhibited (or inhibited to a very low degree) or cell death is not induced as a result of contact with the therapeutic agent, compared to its growth in the absence of contact with the therapeutic agent. The quality of being non-responsive to a therapeutic agent is a highly variable one, with different tumors exhibiting different levels of “non-responsiveness” to a given therapeutic agent, under different conditions.

As used herein, cancers, including tumor cells, refer to neoplastic or hyperplastic cells. Cancers include, but is not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; leukemias and lymphomas such as granulocytic leukemia, monocytic leukemia, lymphocytic leukemia, malignant lymphoma, plasmocytoma, reticulum cell sarcoma, or Hodgkins disease; and tumors of the nervous system including glioma, meningoma, medulloblastoma, schwannoma or epidymoma.

In certain embodiments, 193 responsiveness genes are identified that are differentially expressed between cancer cells sensitive to chemotherapy and those that are less sensitive. These responsiveness genes were identified by comprehensive gene expression profiling on fine needle aspiration specimens from human breast cancers obtained at the time of diagnosis. The set of or subsets of the 193 responsiveness genes may be used to assess the responsiveness of a cancer cell or tumor to a therapy. In certain embodiments, the set or a subset of responsiveness genes, in combination with a prediction algorithm, can be used to identify patients who have a better than average probability to experience a pathologic complete response (pCR) to a therapy, preferably chemotherapy, and more preferably P/FAC therapy. A set or subset of responsiveness genes may include 1, 2, 5, 10, 15, 20, 25, 30, 35, 40, 45, 50, 55, 60, 65, 70, 75, 80, 85, 90, 95, 100, 105, 110, 115, 120, 125, 130, 135, 140 145, 150, 155, 160, 165, 170, 175, 180, 185, 190, or 193 responsiveness gene(s), or any number of responsiveness genes therebetween. The responsiveness genes are set forth in SEQ ID NOs: 1-193. Typically, the genes represented by SEQ ID NO:1-87, 160, 169, and 179 are under-expressed (down regulated) in cancers with complete pathological response, whereas, SEQ ID NO:88-159, 161-168, 170-178, and 180-193 are typically genes that are over-expressed (up-regulated) in cancers with complete pathological response.

I. Analysis of Gene Expression

The present invention provides methods for determining whether a cancer is likely to be sensitive or resistant to a particular therapy or regimen. Although microarray analysis determines the expression levels of thousands of genes in a sample, only a subset of these genes are significantly differentially expressed between cells having different outcomes to therapy. Identifying which of these differentially expressed genes can be used to predict a clinical outcome requires additional analysis.

The genes described in the present invention are genes whose expression varies by a predetermined amount between tumors that are sensitive to a chemotherapy, e.g., P/FAC, versus those that are not responsive or less responsive to a chemotherapy. The following provides detailed descriptions of the genes of interest in the present invention. It is noted that homologs and polymorphic variants of the genes are also contemplated. As described herein, the relative expression of these genes may be measured through nucleic acid hybridization, e.g., microarray analysis. However, other methods of determining expression of the genes are also contemplated. It is also noted that probes for the following genes may be designed using any appropriate fragment of the full lengths of the nucleic acids sequences set forth in SEQ ID NO: 1-193.

Gene expression data may be gathered in any way that is available to one of skill in the art. Typically, gene expression data is obtained by employing an array of probes that hybridize to several, and even thousands or more different transcripts. Such arrays are often classified as microarrays or macroarrays depending on the size of each position on the array.

In one embodiment, the present invention provides methods wherein nucleic acid probes are immobilized on a solid support in an organized array. Oligonucleotides can be bound to a support by a variety of processes, including lithography. It is common in the art to refer to such an array as a “chip.”

In one embodiment, gene expression is assessed by (1) providing a pool of target nucleic acids derived from one or more target genes; (2) hybridizing the nucleic acid sample to an array of probes (including control probes); and (3) detecting nucleic acid hybridization and assessing a relative expression (transcription) level.

TABLE 1
Top 193 Responsiveness Genes

| | | | | T-stat | |
|---|---|---|---|---|---|
| | Accession | | | (MeanCR- | |
| Probe.Set | SEQ ID NO | LocusLink | Name | MeanNR)/se | P-val |
| 203930_s_at | NM_016835.1 | 4137 | Microtubule- | −6.42 | 5.25 × 10⁻⁰⁸ |
| | SEQ ID NO: 1 | | associated protein | | |
| 212745_s_at | AI813772 | 585 | Bardet-Biedl | −6.25 | 9.40 × 10⁻⁰⁸ |
| | SEQ ID NO: 2 | | syndrome 4 | | |
| 203928_x_at | NM_016835.1 | 4137 | Microtubule- | −5.99 | 2.70 × 10⁻⁰⁷ |
| | SEQ ID NO: 1 | | associated protein | | |
| 206401_s_at | J03778.1 | 4137 | Microtubule- | −5.73 | 7.02 × 10⁻⁰⁷ |
| | SEQ ID NO: 3 | | associated protein | | |
| 203929_s_at | NM_016835.1 | 4137 | Microtubule- | −5.52 | 1.26 × 10⁻⁰⁶ |
| | SEQ ID NO: 1 | | associated protein | | |
| 212207_at | AK023837.1 | 23389 | KIAA1025 protein | −5.37 | 2.21 × 10⁻⁰⁶ |
| | SEQ ID NO: 4 | | | | |
| 212046_x_at | X60188.1 | 5595 | Mitogen-activated | −5.33 | 3.43 × 10⁻⁰⁶ |
| | SEQ ID NO: 5 | | protein kinas | | |
| 210469_at | BC002915.1 | 9231 | Discs, large | −5.28 | 3.53 × 10⁻⁰⁶ |
| | SEQ ID NO: 6 | | (Drosophila) homol | | |
| 205074_at | NM_003060.1 | 6584 | Solute carrier | −5.13 | 5.45 × 10⁻⁰⁶ |
| | SEQ ID NO: 7 | | family 22 (organ | | |
| 204509_at | NM_017689.1 | 54837 | Hypothetical protein | −5.02 | 6.15 × 10⁻⁰⁶ |
| | SEQ ID NO: 8 | | FLJ20151 | | |
| 205696_s_at | NM_005264.1 | 2674 | GDNF family | −5.00 | 1.06 × 10⁻⁰⁵ |
| | SEQ ID NO: 9 | | receptor alpha 1 | | |
| 219741_x_at | NM_024762.1 | 79818 | Hypothetical protein | −4.94 | 1.00 × 10⁻⁰⁵ |
| | SEQ ID NO: 10 | | FLJ21603 | | |
| 215616_s_at | AB020683.1 | 23030 | KIAA0876 protein | −4.86 | 1.43 × 10⁻⁰⁵ |
| | SEQ ID NO: 11 | | | | |
| 208945_s_at | NM_003766.1 | 8678 | Beclin 1 (coiled- | −4.86 | 1.48 × 10⁻⁰⁵ |
| | SEQ ID NO: 12 | | coil, myosin-1 | | |
| 217542_at | BE930512 | | ESTs | −4.80 | 1.84 × 10⁻⁰⁵ |
| | SEQ ID NO: 13 | | | | |
| 202204_s_at | AF124145.1 | 267 | Autocrine motility | −4.74 | 2.05 × 10⁻⁰⁵ |
| | SEQ ID NO: 14 | | factor recep | | |
| 204916_at | NM_005855.1 | 10267 | Receptor | −4.70 | 2.92 × 10⁻⁰⁵ |
| | SEQ ID NO: 15 | | (calcitonin) activity | | |
| 218769_s_at | NM_023039.1 | 57763 | Ankyrin repeat, | −4.70 | 2.58 × 10⁻⁰⁵ |
| | SEQ ID NO: 16 | | family A (RFXAN | | |
| 219981_x_at | NM_017961.1 | 55044 | Hypothetical protein | −4.66 | 4.44 × 10⁻⁰⁵ |
| | SEQ ID NO: 17 | | FLJ20813 | | |
| 222131_x_at | BC004327.1 | 89941 | Hypothetical protein | −4.64 | 3.26 × 10⁻⁰⁵ |
| | SEQ ID NO: 18 | | BC014942 | | |
| 213234_at | AB040900.1 | 57613 | KIAA1467 protein | −4.60 | 3.73 × 10⁻⁰⁵ |
| | SEQ ID NO: 19 | | | | |
| 219197_s_at | AI424243 | 57758 | CEGP1 protein | −4.57 | 3.45 × 10⁻⁰⁵ |
| | SEQ ID NO: 20 | | | | |
| 205425_at | NM_005338.3 | 3092 | Huntington | −4.51 | 8.86 × 10⁻⁰⁵ |
| | SEQ ID NO: 21 | | interacting protein | | |
| 213504_at | W63732 | 10980 | COP9 subunit 6 | −4.50 | 4.98 × 10⁻⁰⁵ |
| | SEQ ID NO: 22 | | (MOV34 homolog, | | |
| 201413_at | NM_000414.1 | 3295 | Hydroxysteroid (17- | −4.46 | 5.71 × 10⁻⁰⁵ |
| | SEQ ID NO: 23 | | beta) dehydr | | |
| 203050_at | NM_005657.1 | 7158 | Tumor protein p53 | −4.45 | 7.53 × 10⁻⁰⁵ |
| | SEQ ID NO: 24 | | binding prote | | |
| 212494_at | AB028998.1 | 23371 | KIAA1075 protein | −4.43 | 9.46 × 10⁻⁰⁵ |
| | SEQ ID NO: 25 | | | | |
| 209173_at | AF088867.1 | 10551 | Anterior gradient 2 | −4.41 | 6.36 × 10⁻⁰⁵ |
| | SEQ ID NO: 26 | | homolog (Xe | | |
| 201124_at | AL048423 | 3693 | Integrin, beta 5 | −4.41 | 7.76 × 10⁻⁰⁵ |
| | SEQ ID NO: 27 | | | | |
| 205354_at | NM_000156.3 | 2593 | Guanidinoacetate | −4.39 | 8.11 × 10⁻⁰⁵ |
| | SEQ ID NO: 28 | | N-methyltransf | | |
| 212444_at | AA156240 | | Homo sapiens | −4.37 | 7.71 × 10⁻⁰⁵ |
| | SEQ ID NO: 29 | | cDNA: FLJ22182 | | |
| | | | fis | | |
| 205225_at | NM_000125.1 | 2099 | Estrogen receptor 1 | −4.37 | 8.12 × 10⁻⁰⁵ |
| | SEQ ID NO: 30 | | | | |
| 211000_s_at | AB015706.1 | 3572 | Interleukin 6 signal | −4.36 | 9.16 × 10⁻⁰⁵ |
| | SEQ ID NO: 31 | | transducer | | |
| 204012_s_at | AL529189 | 9836 | KIAA0547 gene | −4.36 | 8.63 × 10⁻⁰⁵ |
| | SEQ ID NO: 32 | | product | | |
| 203682_s_at | NM_002225.2 | 3712 | Isovaleryl | −4.35 | 7.60 × 10⁻⁰⁵ |
| | SEQ ID NO: 33 | | Coenzyme A | | |
| | | | dehydroge | | |
| 220357_s_at | NM_016276.1 | 10110 | Serum/glucocorticoid | −4.35 | 5.94 × 10⁻⁰⁵ |
| | SEQ ID NO: 34 | | regulated | | |
| 216173_at | AK025360.1 | | Homo sapiens | −4.32 | 7.65 × 10⁻⁰⁵ |
| | SEQ ID NO: 35 | | cDNA: FLJ21707 | | |
| | | | fis | | |
| 210230_at | BC003629.1 | 6066 | RNA, U2 small | −4.26 | 9.95 × 10⁻⁰⁵ |
| | SEQ ID NO: 36 | | nuclear | | |
| 219044_at | NM_018271.1 | 55258 | Hypothetical protein | −4.25 | 1.75 × 10⁻⁰⁴ |
| | SEQ ID NO: 37 | | FLJ10916 | | |
| 218761_at | NM_017610.1 | 54778 | Likely ortholog of | −4.23 | 1.35 × 10⁻⁰⁴ |
| | SEQ ID NO: 38 | | mouse Arkadi | | |
| 210826_x_at | AF098533.1 | 5884 | RAD17 homolog | −4.22 | 1.44 × 10⁻⁰⁴ |
| | SEQ ID NO: 39 | | (S. pombe) | | |
| 210831_s_at | L27489.1 | 5733 | Prostaglandin E | −4.22 | 1.07 × 10⁻⁰⁴ |
| | SEQ ID NO: 40 | | receptor 3 (sub | | |
| 211233_x_at | M12674.1 | 2099 | Estrogen receptor 1 | −4.21 | 1.20 × 10⁻⁰⁴ |
| | SEQ ID NO: 41 | | | | |
| 218807_at | NM_006113.2 | 10451 | Vav 3 oncogene | −4.20 | 1.46 × 10⁻⁰⁴ |
| | SEQ ID NO: 42 | | | | |
| 210129_s_at | AF078842.1 | 26140 | DKFZP434B103 | −4.19 | 1.09 × 10⁻⁰⁴ |
| | SEQ ID NO: 43 | | protein | | |
| 39313_at | AB002342 | 65125 | Protein kinase, | −4.19 | 1.23 × 10⁻⁰⁴ |
| | SEQ ID NO: 44 | | lysine deficien | | |
| 213245_at | AL120173 | | Homo sapiens | −4.18 | 1.43 × 10⁻⁰⁴ |
| | SEQ ID NO: 45 | | cDNA FLJ30781 | | |
| | | | fis, | | |
| 214053_at | AW772192 | | Homo sapiens clone | −4.18 | 1.51 × 10⁻⁰⁴ |
| | SEQ ID NO: 46 | | 23736 mRNA s | | |
| 205352_at | NM_005025.1 | 5274 | Serine (or cysteine) | −4.17 | 1.47 × 10⁻⁰⁴ |
| | SEQ ID NO: 47 | | proteinase | | |
| 213623_at | NM_007054.1 | 11127 | Kinesin family | −4.15 | 1.88 × 10⁻⁰⁴ |
| | SEQ ID NO: 48 | | member 3A | | |
| 215304_at | U79293.1 | | Human clone 23948 | −4.13 | 1.40 × 10⁻⁰⁴ |
| | SEQ ID NO: 49 | | mRNA sequence | | |
| 203009_at | NM_005581.1 | 4059 | Lutheran blood | −4.13 | 1.80 × 10⁻⁰⁴ |
| | SEQ ID NO: 50 | | group (Auberger | | |
| 218692_at | NM_017786.1 | 55638 | Hypothetical protein | −4.13 | 1.76 × 10⁻⁰⁴ |
| | SEQ ID NO: 51 | | FLJ20366 | | |
| 218976_at | NM_021800.1 | 56521 | J domain containing | −4.12 | 1.76 × 10⁻⁰⁴ |
| | SEQ ID NO: 52 | | protein 1 | | |
| 201405_s_at | NM_006833.1 | 10980 | COP9 subunit 6 | −4.11 | 1.63 × 10⁻⁰⁴ |
| | SEQ ID NO: 53 | | (MOV34 homolog, | | |
| 202168_at | NM_003187.1 | 6880 | TAF9 RNA | −4.11 | 2.01 × 10⁻⁰⁴ |
| | SEQ ID NO: 54 | | polymerase II, | | |
| | | | TATA bo | | |
| 216109_at | AK025348.1 | | Homo sapiens | −4.11 | 1.77 × 10⁻⁰⁴ |
| | SEQ ID NO: 55 | | cDNA: FLJ21695 | | |
| | | | fis | | |
| 219051_x_at | NM_024042.1 | 79006 | Hypothetical protein | −4.10 | 2.34 × 10⁻⁰⁴ |
| | SEQ ID NO: 56 | | MGC2601 | | |
| 210908_s_at | AB055804.1 | 5204 | Prefoldin 5 | −4.09 | 1.71 × 10⁻⁰⁴ |
| | SEQ ID NO: 57 | | | | |
| 221728_x_at | AK025198.1 | | Homo sapiens | −4.07 | 2.11 × 10⁻⁰⁴ |
| | SEQ ID NO: 58 | | cDNA FLJ30298 | | |
| | | | fis, | | |
| 203187_at | NM_001380.1 | 1793 | Dedicator of cytokinesis 1 | −4.06 | 2.22 × 10⁻⁰⁴ |
| | SEQ ID NO59 | | | | |
| 212660_at | AI735639 | 23338 | KIAA0239 protein | −4.04 | 2.56 × 10⁻⁰⁴ |
| | SEQ ID NO: 60 | | | | |
| 212956_at | AB020689.1 | 23158 | KIAA0882 protein | −4.01 | 2.27 × 10⁻⁰⁴ |
| | SEQ ID NO: 61 | | | | |
| 217838_s_at | NM_016337.1 | 51466 | RNB6 | −4.01 | 2.14 × 10⁻⁰⁴ |
| | SEQ ID NO: 62 | | | | |
| 218621_at | NM_016173.1 | 51409 | HEMK homolog | −4.01 | 1.92 × 10⁻⁰⁴ |
| | SEQ ID NO: 63 | | 7 kb | | |
| 201681_s_at | AB011155.1 | 9231 | Discs, large | −4.01 | 2.49 × 10⁻⁰⁴ |
| | SEQ ID NO: 64 | | (Drosophila) homol | | |
| 209884_s_at | AF047033.1 | 9497 | Solute carrier | −4.00 | 2.98 × 10⁻⁰⁴ |
| | SEQ ID NO: 65 | | family 4, sodium | | |
| 201557_at | NM_014232.1 | 6844 | Vesicle-associated | −3.99 | 2.23 × 10⁻⁰⁴ |
| | SEQ ID NO: 66 | | membrane pro | | |
| 219338_s_at | NM_017691.1 | 54839 | Hypothetical protein | −3.99 | 2.94 × 10⁻⁰⁴ |
| | SEQ ID NO: 67 | | FLJ20156 | | |
| 217828_at | NM_024755.1 | 79811 | Hypothetical protein | −3.98 | 2.42 × 10⁻⁰⁴ |
| | SEQ ID NO: 68 | | FLJ13213 | | |
| 209339_at | U76248.1 | 6478 | Seven in absentia | −3.98 | 2.26 × 10⁻⁰⁴ |
| | SEQ ID NO: 69 | | homolog 2 (Dr | | |
| 214218_s_at | AV699347 | | Homo sapiens | −3.97 | 2.82 × 10⁻⁰⁴ |
| | SEQ ID NO: 70 | | cDNA FLJ30298 | | |
| | | | fis, | | |
| 221643_s_at | AF016005.1 | 473 | Arginine-glutamic | −3.96 | 2.57 × 10⁻⁰⁴ |
| | SEQ ID NO: 71 | | acid dipeptid | | |
| 218211_s_at | NM_024101.1 | 79083 | Melanophilin | −3.95 | 3.05 × 10⁻⁰⁴ |
| | SEQ ID NO: 72 | | | | |
| 221483_s_at | AF084555.1 | 10776 | Cyclic AMP | −3.95 | 2.83 × 10⁻⁰⁴ |
| | SEQ ID NO: 73 | | phosphoprotein, 19 k | | |
| 211864_s_at | AF207990.1 | 26509 | Fer-1-like 3, | −3.92 | 3.29 × 10⁻⁰⁴ |
| | SEQ ID NO: 74 | | myoferlin (C. ele | | |
| 202392_s_at | NM_014338.1 | 23761 | Phosphatidylserine | −3.92 | 4.33 × 10⁻⁰⁴ |
| | SEQ ID NO: 75 | | decarboxylas | | |
| 214164_x_at | BF752277 | 164 | Adaptor-related | −3.91 | 3.52 × 10⁻⁰⁴ |
| | SEQ ID NO: 76 | | protein complex | | |
| 204862_s_at | NM_002513.1 | 4832 | Non-metastatic cells | −3.91 | 3.55 × 10⁻⁰⁴ |
| | SEQ ID NO: 77 | | 3, protein | | |
| 215552_s_at | AI073549 | 2099 | Estrogen receptor 1 | −3.91 | 3.33 × 10⁻⁰⁴ |
| | SEQ ID NO: 78 | | | | |
| 211235_s_at | AF258450.1 | 2099 | Estrogen receptor 1 | −3.90 | 3.13 × 10⁻⁰⁴ |
| | SEQ ID NO: 79 | | | | |
| 210833_at | AL031429 | 5733 | Prostaglandin E | −3.89 | 3.06 × 10⁻⁰⁴ |
| | SEQ ID NO: 80 | | receptor 3 (sub | | |
| 204660_at | NM_005262.1 | 2671 | Growth factor, | −3.89 | 2.79 × 10⁻⁰⁴ |
| | SEQ ID NO: 81 | | augmenter of liv | | |
| 211234_x_at | AF258449.1 | 2099 | Estrogen receptor 1 | −3.89 | 3.10 × 10⁻⁰⁴ |
| | SEQ ID NO: 82 | | | | |
| 201508_at | NM_001552.1 | 3487 | Insulin-like growth | −3.88 | 4.04 × 10⁻⁰⁴ |
| | SEQ ID NO: 83 | | factor bind | | |
| 213527_s_at | AI350500 | 146542 | Similar to | −3.85 | 4.33 × 10⁻⁰⁴ |
| | SEQ ID NO: 84 | | hypothetical protein | | |
| 202048_s_at | NM_014292.1 | 23466 | Chromobox | −3.84 | 4.15 × 10⁻⁰⁴ |
| | SEQ ID NO: 85 | | homolog 6 | | |
| 206794_at | NM_005235.1 | 2066 | v-erb-a | −3.84 | 3.87 × 10⁻⁰⁴ |
| | SEQ ID NO: 86 | | erythroblastic | | |
| | | | leukemia | | |
| 201798_s_at | NM_013451.1 | 26509 | Fer-1-like 3, | −3.83 | 4.44 × 10⁻⁰⁴ |
| | SEQ ID NO: 87 | | myoferlin (C. ele | | |
| 213523_at | AI671049 | 898 | Cyclin E1 | 3.81 | 4.14 × 10⁻⁰⁴ |
| | SEQ ID NO: 88 | | | | |
| 209050_s_at | AI421559 | 5900 | Ral guanine | 3.83 | 4.07 × 10⁻⁰⁴ |
| | SEQ ID NO: 89 | | nucleotide dissocia | | |
| 217294_s_at | U88968.1 | 2023 | Enolase 1, (alpha) | 3.84 | 4.48 × 10⁻⁰⁴ |
| | SEQ ID NO: 90 | | | | |
| 201555_at | NM_002388.2 | 4172 | MCM3 | 3.84 | 4.41 × 10⁻⁰⁴ |
| | SEQ ID NO: 91 | | minichromosome | | |
| | | | maintenance | | |
| 201030_x_at | NM_002300.1 | 3945 | Lactate | 3.85 | 3.85 × 10⁻⁰⁴ |
| | SEQ ID NO: 92 | | dehydrogenase B | | |
| 202912_at | NM_001124.1 | 133 | Adrenomedullin | 3.86 | 3.59 × 10⁻⁰⁴ |
| | SEQ ID NO: 93 | | | | |
| 204050_s_at | NM_001833.1 | 1211 | Clathrin, light | 3.88 | 3.97 × 10⁻⁰⁴ |
| | SEQ ID NO: 94 | | polypeptide (Lc | | |
| 202342_s_at | NM_015271.1 | 23321 | Tripartite motif- | 3.88 | 4.43 × 10⁻⁰⁴ |
| | SEQ ID NO: 95 | | containing 2 | | |
| 209393_s_at | AF047695.1 | 9470 | Eukaryotic | 3.89 | 4.21 × 10⁻⁰⁴ |
| | SEQ ID NO: 96 | | translation initiati | | |
| 219774_at | NM_019044.1 | 54520 | Hypothetical protein | 3.93 | 3.86 × 10⁻⁰⁴ |
| | SEQ ID NO: 97 | | FLJ10996 | | |
| 204162_at | NM_006101.1 | 10403 | Highly expressed in | 3.93 | 2.94 × 10⁻⁰⁴ |
| | SEQ ID NO: 98 | | cancer, ric | | |
| 216237_s_at | AA807529 | 4174 | MCM5 | 3.96 | 2.84 × 10⁻⁰⁴ |
| | SEQ ID NO: 99 | | minichromosome | | |
| | | | maintenance | | |
| 214581_x_at | BE568134 | 27242 | Tumor necrosis | 3.99 | 3.07 × 10⁻⁰⁴ |
| | SEQ ID NO: 100 | | factor receptor | | |
| 209408_at | U63743.1 | 11004 | Kinesin-like 6 | 3.99 | 2.23 × 10⁻⁰⁴ |
| | SEQ ID NO: 101 | | (mitotic centrom | | |
| 208370_s_at | NM_004414.2 | 1827 | Down syndrome | 4.02 | 2.94 × 10⁻⁰⁴ |
| | SEQ ID NO: 102 | | critical region g | | |
| 203744_at | NM_005342.1 | 3149 | High-mobility | 4.02 | 2.02 × 10⁻⁰⁴ |
| | SEQ ID NO: 103 | | group box 3 | | |
| 209575_at | BC001903.1 | 3588 | Interleukin 10 | 4.03 | 2.84 × 10⁻⁰⁴ |
| | SEQ ID NO: 104 | | receptor, beta | | |
| 200934_at | NM_003472.1 | 7913 | DEK oncogene | 4.05 | 2.54 × 10⁻⁰⁴ |
| | SEQ ID NO: 105 | | (DNA binding) | | |
| 202341_s_at | AA149745 | 23321 | Tripartite motif- | 4.06 | 2.87 × 10⁻⁰⁴ |
| | SEQ ID NO: 106 | | containing 2 | | |
| 200996_at | NM_005721.2 | 10096 | ARP3 actin-related | 4.06 | 2.42 × 10⁻⁰⁴ |
| | SEQ ID NO: 107 | | protein 3 ho | | |
| 206392_s_at | NM_002888.1 | 5918 | Retinoic acid | 4.06 | 2.28 × 10⁻⁰⁴ |
| | SEQ ID NO: 108 | | receptor responde | | |
| 206391_at | NM_002888.1 | 5918 | Retinoic acid | 4.07 | 2.52 × 10⁻⁰⁴ |
| | SEQ ID NO: 109 | | receptor responde | | |
| 201797_s_at | NM_006295.1 | 7407 | Valyl-tRNA | 4.07 | 2.17 × 10⁻⁰⁴ |
| | SEQ ID NO: 110 | | synthetase 2 | | |
| 209358_at | AF118094.1 | 6882 | TAF11 RNA | 4.07 | 2.34 × 10⁻⁰⁴ |
| | SEQ ID NO: 111 | | polymerase II, | | |
| | | | TATA b | | |
| 209201_x_at | L01639.1 | 7852 | Chemokine (C—X—C | 4.09 | 2.80 × 10⁻⁰⁴ |
| | SEQ ID NO: 112 | | motif) recepto | | |
| 209016_s_at | BC002700.1 | 3855 | Keratin 7 | 4.14 | 1.69 × 10⁻⁰⁴ |
| | SEQ ID NO: 113 | | | | |
| 221957_at | BF939522 | 5165 | Pyruvate | 4.15 | 2.22 × 10⁻⁰⁴ |
| | SEQ ID NO: 114 | | dehydrogenase | | |
| | | | kinase, | | |
| 218350_s_at | NM_015895.1 | 51053 | Geminin, DNA | 4.16 | 1.64 × 10⁻⁰⁴ |
| | SEQ ID NO: 115 | | replication inhibi | | |
| 201897_s_at | NM_001826.1 | 84722 | p53-regulated | 4.21 | 1.36 × 10⁻⁰⁴ |
| | SEQ ID NO: 116 | | DDA3 | | |
| 209642_at | AF043294.2 | 699 | BUB1 budding | 4.22 | 1.22 × 10⁻⁰⁴ |
| | SEQ ID NO: 117 | | uninhibited by ben | | |
| 201930_at | NM_005915.2 | 4175 | MCM6 | 4.23 | 1.16 × 10⁻⁰⁴ |
| | SEQ ID NO: 118 | | minichromosome | | |
| | | | maintenance | | |
| 202870_s_at | NM_001255.1 | 991 | CDC20 cell division | 4.23 | 1.07 × 10⁻⁰⁴ |
| | SEQ ID NO: 119 | | cycle 20 ho | | |
| 221485_at | NM_004776.1 | 9334 | UDP- | 4.26 | 1.08 × 10⁻⁰⁴ |
| | SEQ ID NO: 120 | | Gal: betaGlcNAc | | |
| | | | beta 1,4-ga | | |
| 211919_s_at | AF348491.1 | 7852 | Chemokine (C—X—C | 4.27 | 1.61 × 10⁻⁰⁴ |
| | SEQ ID NO: 121 | | motif) recepto | | |
| 218887_at | NM_015950.1 | 51069 | Mitochondrial | 4.27 | 8.93 × 10⁻⁰⁵ |
| | SEQ ID NO: 122 | | ribosomal protein | | |
| 216295_s_at | X81636.1 | | H. sapiens clathrin | 4.28 | 1.17 × 10⁻⁰⁴ |
| | SEQ ID NO: 123 | | light chain | | |
| 218726_at | NM_018410.1 | 55355 | Hypothetical protein | 4.28 | 1.19 × 10⁻⁰⁴ |
| | SEQ ID NO: 124 | | DKFZp762E1 | | |
| 204989_s_at | BF305661 | 3691 | Integrin, beta 4 | 4.30 | 1.01 × 10⁻⁰⁴ |
| | SEQ ID NO: 125 | | | | |
| 221872_at | AI669229 | 5918 | Retinoic acid | 4.31 | 1.12 × 10⁻⁰⁴ |
| | SEQ ID NO: 126 | | receptor responde | | |
| 206746_at | NM_001195.2 | 631 | Beaded filament | 4.32 | 9.33 × 10⁻⁰⁵ |
| | SEQ ID NO: 127 | | structural prot | | |
| 201231_s_at | NM_001428.1 | 2023 | Enolase 1, (alpha) | 4.42 | 5.76 × 10⁻⁰⁵ |
| | SEQ ID NO: 128 | | | | |
| 204203_at | NM_001806.1 | 1054 | CCAAT/enhancer | 4.42 | 6.44 × 10⁻⁰⁵ |
| | SEQ ID NO: 129 | | binding protein | | |
| 211555_s_at | AF020340.1 | 2983 | Guanylate cyclase | 4.47 | 5.11 × 10⁻⁰⁵ |
| | SEQ ID NO: 130 | | 1, soluble, b | | |
| 202200_s_at | NM_003137.1 | 6732 | SFRS protein kinase 1 | 4.47 | 5.17 × 10⁻⁰⁵ |
| | SEQ ID NO: 131 | | | | |
| 213101_s_at | Z78330 | | Homo sapiens | 4.49 | 7.76 × 10⁻⁰⁵ |
| | SEQ ID NO: 132 | | mRNA; cDNA | | |
| | | | DKFZp68 | | |
| 204600_at | NM_004443.1 | 2049 | EphB3 | 4.51 | 5.81 × 10⁻⁰⁵ |
| | SEQ ID NO: 133 | | | | |
| 212689_s_at | AA524505 | 55818 | Zinc finger protein | 4.52 | 5.10 × 10⁻⁰⁵ |
| | SEQ ID NO: 134 | | | | |
| 209773_s_at | BC001886.1 | 6241 | Ribonucleotide | 4.55 | 3.18 × 10⁻⁰⁵ |
| | SEQ ID NO: 135 | | reductase M2 pol | | |
| 204962_s_at | NM_001809.2 | 1058 | Centromere protein | 4.62 | 3.00 × 10⁻⁰⁵ |
| | SEQ ID NO: 136 | | A, 17 kDa | | |
| 211519_s_at | AY026505.1 | 11004 | Kinesin-like 6 | 4.62 | 2.41 × 10⁻⁰⁵ |
| | SEQ ID NO: 137 | | (mitotic centrom | | |
| 204825_at | NM_014791.1 | 9833 | Maternal embryonic | 4.73 | 2.45 × 10⁻⁰⁵ |
| | SEQ ID NO: 138 | | leucine zipp | | |
| 203287_at | NM_005558.1 | 3898 | Ladinin 1 | 4.74 | 2.06 × 10⁻⁰⁵ |
| | SEQ ID NO: 139 | | | | |
| 204913_s_at | AI360875 | 6664 | SRY (sex | 4.77 | 2.44 × 10⁻⁰⁵ |
| | SEQ ID NO: 140 | | determining region | | |
| | | | Y)- | | |
| 217028_at | AJ224869 | | | 4.82 | 2.56 × 10⁻⁰⁵ |
| | SEQ ID NO: 141 | | | | |
| 204750_s_at | BF196457 | 1824 | Desmocollin 2 | 4.84 | 1.78 × 10⁻⁰⁵ |
| | SEQ ID NO: 142 | | | | |
| 216222_s_at | AI561354 | 4651 | Myosin X | 4.84 | 1.93 × 10⁻⁰⁵ |
| | SEQ ID NO: 143 | | | | |
| 1438_at | X75208 | 2049 | EphB3 | 5.02 | 9.02 × 10⁻⁰⁶ |
| | SEQ ID NO: 144 | | | | |
| 203693_s_at | NM_001949.2 | 1871 | E2F transcription | 5.17 | 4.83 × 10⁻⁰⁶ |
| | SEQ ID NO: 145 | | factor 3 | | |
| 205548_s_at | NM_006806.1 | 10950 | BTG family, | 5.64 | 1.96 × 10⁻⁰⁶ |
| | SEQ ID NO: 146 | | member 3 | | |
| 201976_s_at | NM_012334.1 | 4651 | Myosin X | 5.68 | 8.74 × 10⁻⁰⁷ |
| | SEQ ID NO: 147 | | | | |
| 213134_x_at | AI765445 | 10950 | BTG family, | 5.76 | 1.31 × 10⁻⁰⁶ |
| | SEQ ID NO: 148 | | member 3 | | |
| 40016_g_at | AB002301 | 23227 | KIAA0303 protein | 4.26 | 1.071 × 10⁻⁰⁴ |
| | SEQ ID NO: 149 | | | | |
| 206352_s_at | AB013818 | 5192 | peroxisome | 4.28 | 5.79 × 10⁻⁰⁵ |
| | SEQ ID NO: 150 | | biogenesis factor 10 | | |
| 205074_at | AB015050 | 6584 | solute carrier family | 4.64 | 2.24 × 10⁻⁰⁵ |
| | SEQ ID NO: 151 | | 22 member 5 | | |
| 213527_s_at | AC002310 | 146542 | similar to | 4.62 | 3.16 × 10⁻⁰⁵ |
| | SEQ ID NO: 152 | | hypothetical protein | | |
| | | | MGC13138 | | |
| 216835_s_at | AF035299 | 1796 | docking protein 1, | 4.44 | 3.32 × 10⁻⁰⁵ |
| | SEQ ID NO: 153 | | 62 kDa | | |
| 209617_s_at | AF035302 | 1501 | catenin (cadherin- | 5.16 | 1.7 × 10⁻⁰⁶ |
| | SEQ ID NO: 154 | | associated protein), | | |
| | | | delta 2 (neural | | |
| | | | plakophilin-related | | |
| | | | arm-repeat protein) | | |
| 208945_s_at | AF139131 | 8678 | beclin 1 (coiled- | 5.61 | 5.0 × 10⁻⁰⁷ |
| | SEQ ID NO: 155 | | coil, myosin-like | | |
| | | | BCL2 interacting | | |
| | | | protein) | | |
| 222275_at | AI039469 | 10884 | mitochondrial | 4.51 | 2.16 × 10⁻⁰⁵ |
| | SEQ ID NO: 156 | | ribosomal protein | | |
| | | | S30 | | |
| 203929_s_at | AI056359 | 4137 | microtubule- | 6.60 | 0.0 × 10⁻⁰⁴ |
| | SEQ ID NO: 157 | | associated protein | | |
| | | | tau | | |
| 215552_s_at | AI073549 | 2099 | Estrogen receptor 1 | 4.51 | 2.51 × 10⁻⁰⁵ |
| | SEQ ID NO: 158 | | | | |
| 212956_at | AI348094 | 23158 | KIAA0882 protein | 4.40 | 7.0 × 10⁻⁰⁵ |
| | SEQ ID NO: 159 | | | | |
| 204913_s_at | AI360875 | 6664 | SRY (sex | −4.45 | 9.92 × 10⁻⁰⁵ |
| | SEQ ID NO: 160 | | determining region | | |
| | | | Y)-box 11 | | |
| 213855_s_at | AI500366 | 3991 | lipase, hormone- | 4.17 | 1.08 × 10⁻⁰⁴ |
| | SEQ ID NO: 161 | | sensitive | | |
| 212239_at | AI680192 | 5295 | phosphoinositide-3- | 4.36 | 4.71 × 10⁻⁰⁵ |
| | SEQ ID NO: 162 | | kinase, regulatory | | |
| | | | subunit, polypeptide | | |
| | | | 1 (p85 alpha) | | |
| 203928_x_at | AI870749 | 4137 | microtubule- | 5.91 | 8 × 10⁻⁰⁸ |
| | SEQ ID NO: 163 | | associated protein | | |
| | | | tau | | |
| 214124_x_at | AL043487 | 11116 | FGFR1 oncogene | 5.18 | 3.1 × 10⁻⁰⁶ |
| | SEQ ID NO: 164 | | partner | | |
| 212195_at | AL049265 | — | MRNA; cDNA | 4.25 | 1.11 × 10⁻⁰⁴ |
| | SEQ ID NO: 165 | | DKFZp564F053 | | |
| 210222_s_at | BC000314 | 6252 | reticulon 1 | 4.08 | 1.07 × 10⁻⁰⁴ |
| | SEQ ID NO: 166 | | | | |
| 210958_s_at | BC003646 | 23227 | KIAA0303 protein | 4.43 | 4.26 × 10⁻⁰⁵ |
| | SEQ ID NO: 167 | | | | |
| 204863_s_at | BE856546 | 3572 | interleukin 6 signal | 4.28 | 8.20 × 10⁻⁰⁵ |
| | SEQ ID NO: 168 | | transducer (gp130, | | |
| | | | oncostatin M | | |
| | | | receptor) | | |
| 213911_s_at | BF718636 | 3015 | H2A histone family, | −4.16 | 1.10 × 10⁻⁰⁴ |
| | SEQ ID NO: 169 | | member Z | | |
| 212207_at | BG426689 | 23389 | thyroid hormone | 6.06 | 1.0 × 10⁻⁰⁷ |
| | SEQ ID NO: 170 | | receptor associated | | |
| | | | protein 2 | | |
| 209696_at | D26054 | 2203 | fructose-1,6- | 4.29 | 9.21 × 10⁻⁰⁵ |
| | SEQ ID NO: 171 | | bisphosphatase 1 | | |
| 209443_at | J02639 | 5104 | serine (or cysteine) | 4.21 | 6.95 × 10⁻⁰⁵ |
| | SEQ ID NO: 172 | | proteinase inhibitor, | | |
| | | | clade A (alpha-1 | | |
| | | | antiproteinase, | | |
| | | | antitrypsin), | | |
| | | | member 5 | | |
| 202862_at | NM_000137 | 2184 | fumarylacetoacetate | 4.34 | 5.59 × 10⁻⁰⁵ |
| | SEQ ID NO: 173 | | hydrolase | | |
| | | | (fumarylacetoacetase) | | |
| 214440_at | NM_000662 | 9 | N-acetyltransferase | 4.24 | 6.75 × 10⁻⁰⁵ |
| | SEQ ID NO: 174 | | 1 (arylamine N- | | |
| | | | acetyltransferase) | | |
| 208305_at | NM_000926 | 5241 | progesterone | 4.15 | 8.19 × 10⁻⁰⁵ |
| | SEQ ID NO: 175 | | receptor | | |
| 202204_s_at | NM_001144 | 267 | autocrine motility | 5.28 | 1.29 × 10⁻⁰⁶ |
| | SEQ ID NO: 176 | | factor receptor | | |
| 204862_s_at | NM_002513 | 4832 | non-metastatic cells | 4.30 | 8.95 × 10⁻⁰⁵ |
| | SEQ ID NO: 177 | | 3, protein expressed | | |
| | | | in | | |
| 202641_at | NM_004311 | 403 | ADP-ribosylation | 4.24 | 9.46 × 10⁻⁰⁵ |
| | SEQ ID NO: 178 | | factor-like 3 | | |
| 200896_x_at | NM_004494 | 3068 | hepatoma-derived | −4.87 | 1.38 × 10⁻⁰⁵ |
| | SEQ ID NO: 179 | | growth factor (high- | | |
| | | | mobility group | | |
| | | | protein 1-like) | | |
| 203071_at | NM_004636 | 7869 | sema domain, | 4.65 | 1.63 × 10⁻⁰⁵ |
| | SEQ ID NO: 180 | | immunoglobulin | | |
| | | | domain (Ig), short | | |
| | | | basic domain, | | |
| | | | secreted, | | |
| | | | (semaphorin) 3B | | |
| 205012_s_at | NM_005326 | 3029 | hydroxyacylglutathi | 4.60 | 3.62 × 10⁻⁰⁵ |
| | SEQ ID NO: 181 | | one hydrolase | | |
| 204916_at | NM_005855 | 10267 | receptor (calcitonin) | 5.47 | 5.10 × 10⁻⁰⁷ |
| | SEQ ID NO: 182 | | activity modifying | | |
| | | | protein 1 | | |
| 204792_s_at | NM_014714 | 9742 | KIAA0590 gene | 4.14 | 1.12 × 10⁻⁰⁴ |
| | SEQ ID NO: 183 | | product | | |
| 208202_s_at | NM_015288 | 23338 | PHD finger protein | 4.18 | 1.08 × 10⁻⁰⁴ |
| | SEQ ID NO: 184 | | 15 | | |
| 217770_at | NM_015937 | 51604 | phosphatidylinositol | 4.33 | 5.43 × 10⁻⁰⁵ |
| | SEQ ID NO: 185 | | glycan, class T | | |
| 218671_s_at | NM_016311 | 93974 | ATPase inhibitory | 4.18 | 9.04 × 10⁻⁰⁵ |
| | SEQ ID NO: 186 | | factor 1 | | |
| 219872_at | NM_016613 | 51313 | hypothetical protein | 4.10 | 1.03 × 10⁻⁰⁴ |
| | SEQ ID NO: 187 | | DKFZp434L142 | | |
| 219197_s_at | NM_020974 | 57758 | signal peptide, CUB | 5.43 | 6.8 × 10⁻⁰⁷ |
| | SEQ ID NO: 188 | | domain, EGF-like 2 | | |
| 203485_at | NM_021136 | 6252 | reticulon 1 | 4.18 | 7.56 × 10⁻⁰⁵ |
| | SEQ ID NO: 189 | | | | |
| 206936_x_at | NM_022335 | 4718 | NADH | 4.28 | 6.46 × 10⁻⁰⁵ |
| | SEQ ID NO: 190 | | dehydrogenase | | |
| | | | (ubiquinone) 1, | | |
| | | | subcomplex | | |
| | | | unknown, 2, | | |
| | | | 14.5 kDa | | |
| 220540_at | NM_022358 | 60598 | potassium channel, | 4.68 | 1.32 × 10⁻⁰⁵ |
| | SEQ ID NO: 191 | | subfamily K, | | |
| | | | member 15 | | |
| 219438_at | NM_024522 | 79570 | hypothetical protein | 4.82 | 6.68 × 10⁻⁰⁶ |
| | SEQ ID NO: 192 | | FLJ12650 | | |
| 205696_s_at | U97144 | 2674 | GDNF family | 4.89 | 7.15 × 10⁻⁰⁶ |
| | SEQ ID NO: 193 | | receptor alpha 1 | | |

A. Tau Gene Encodes a Microtubule-Associated Protein

Previous reports indicate that Tau promotes assembly and stabilization of microtubules similar to paclitaxel but with lower affinity and in a reversible manner (Drubin and Kirschner, 1986; Al-Bassam et al., 2002). The inventors examined if Tau could reduce paclitaxel-induced microtubule polymerization and found that pre-incubation of tubulin with Tau substantially reduced polymerization caused by paclitaxel. This could occur through substrate depletion or direct inhibition of paclitaxel binding to tubulin. The presence of Tau reduces the binding of fluorescent paclitaxel to tubulin in vitro and also reduces the accumulation of fluorescent paclitaxel in breast cancer cells in culture. These results demonstrate that Tau partially protects cells from paclitaxel-induced microtubule polymerization and subsequent cell death by competing with paclitaxel for binding to tubulin. Tau is able to bind to both at the outer surface and to the inner, luminal surface of microtubules. The luminal surface contains the paclitaxel binding sites. Kar et al. (2003) have reported that Tau stabilizes microtubules in a similar way to paclitaxel, and it may be the natural substrate that binds to the ‘paclitaxel’ pocket in β-tubulin.

Other investigators have reported that under different experimental circumstances Tau may enhance cooperative binding of paclitaxel to microtubules (Diaz et al., 2003). In all of these reports, paclitaxel exposure preceded Tau exposure and this could account for the different results. When the function of Tau is studied on paclitaxel-stabilized microtubules, Tau binds to the outer surface of tubulin rather than to the inner surface and enhances polymerization by paclitaxel (Al-Bassam et al., 2002; Chau et al., 1998).

As described herein, Tau or a gene encoding Tau is a marker of sensitivity to paclitaxel-containing chemotherapy, it is also clear that many tumors despite low Tau expression are not fully sensitive to treatment. Tau has a strong negative correlation with pathological CR. Around 50% of patients with low Tau expression had residual cancer suggesting frequent additional pathways of resistance. A few tumors with high Tau expression (14%) also experienced complete pathologic response. These observations are consistent with the commonly held belief that response and resistance to chemotherapy are multifactorial processes involving drug transport, drug metabolism, and alterations in drug targets and in pro- and anti-apoptotic pathways (Horwitz et al., 1993; Orr et al., 2003).

Tau could be used as a marker to identify the subset of patients who benefit from paclitaxel-containing therapy and could also serve as a target to modulate response to paclitaxel. The association between Tau and pathological CR has been validated using immunohistochemistry in an independent patient population. Down regulation of Tau expression is also shown herein to increase the sensitivity of breast cancer cells to paclitaxel, and also used to describe a mechanism for the sensitization to chemotherapy.

Low expression of microtubule-associated protein Tau within the tumor at the time of diagnosis was significantly associated with complete pathologic response. The inventors have validated this association at the protein level on an independent set of patients (n=122) using immunohistochemistry. Low Tau expression was shown to be not only a marker of response but it causes sensitivity to paclitaxel in vitro. Down regulation or reduction in the expression of Tau with, for example, siRNA in cancer cells increases sensitivity to paclitaxel, but not to epirubicin. Tau partially protects cells from paclitaxel induced apoptosis by reducing paclitaxel binding to tubulin and reducing paclitaxel induced microtubule polymerization. These observations suggest that Tau is a clinically useful predictor of benefit from paclitaxel-containing adjuvant chemotherapy for breast cancer and that inhibition of Tau function sensitizes cells to paclitaxel.

As described herein, low levels of Tau mRNA expression as measured by, but not limited to, cDNA microarrays or Tau protein expression detected by immunohistochemistry, are associated with higher rates of pathologic CR to P/FAC pre-operative chemotherapy for stage I-III breast cancer. This association was observed in two independent patient cohorts treated with essentially identical chemotherapy regimens. Pathologic CR in this context means complete eradication of the invasive cancer from the breast and lymph nodes by chemotherapy and has consistently been associated with excellent long-term survival that is independent of other tumor characteristics. The results indicate that assessment of Tau expression helps to identify patients at the time of diagnosis who have highly P/FAC sensitive tumors and therefore should receive this regimen if adjuvant or neoadjuvant chemotherapy is indicated.

Low Tau expression is associated with known clinicopathological predictors of response to chemotherapy such as ER-negative status and high nuclear grade. However, in contrast to these predictors that are not treatment regimen-specific, low Tau may predict extreme sensitivity to a particular drug, paclitaxel. Since Tau is a microtubule associated protein, Tau has a mechanistic role in determining cellular response to paclitaxel, which is a microtubule poison. The demonstration that down regulation of Tau by siRNA in breast cancer cells increases their sensitivity to paclitaxel but not to epirubicin suggests a direct role for Tau in determining response to this drug. Guise et al. (1999) have examined apoptosis induced by paclitaxel in the neuroblastoma SK-N-SH cell line with a special focus on Tau protein and have reported that treatment with retinoic acid increased Tau expression and decreased sensitivity to paclitaxel.

Tau represents a paclitaxel-specific predictor of sensitivity. This molecule may be used to identify patients with newly diagnosed breast cancer who require paclitaxel containing chemotherapy to maximize their chance of cure. Tau is also a potential therapeutic target because inhibition of its function increases sensitivity to paclitaxel.

B. Providing a Nucleic Acid Sample

One of skill in the art will appreciate that in order to assess the transcription level (and thereby the expression level) of a gene or genes, it is desirable to provide a nucleic acid sample derived from the mRNA transcript(s). As used herein, a nucleic acid derived from a mRNA transcript refers to a nucleic acid for whose synthesis the mRNA transcript or a subsequence thereof has ultimately served as a template. Thus, a cDNA reverse transcribed from an mRNA, an RNA transcribed from the cDNA, a DNA amplified from the cDNA, an RNA transcribed from the amplified DNA, and the like, are all derived from the mRNA transcript. Detection of such derived products is indicative of the presence and abundance of the original transcript in a sample. Thus, suitable samples include, but are not limited to, mRNA transcripts of the gene or genes, cDNA reverse transcribed from the mRNA, cRNA transcribed from the cDNA, and the like.

Where it is desired to quantify the transcription level of one or more genes in a sample, the concentration of the mRNA transcript(s) of the gene or genes is proportional to the transcription level of that gene. Similarly, it is preferred that the hybridization signal intensity be proportional to the amount of hybridized nucleic acid. As described herein, controls can be run to correct for variations introduced in sample preparation and hybridization.

In one embodiment, a nucleic acid sample is the total mRNA isolated from a biological sample. The term “biological sample,” as used herein, refers to a sample obtained from an organism or from components (e.g., cells) of an organism, including diseased tissue such as a tumor, a neoplasia or a hyperplasia. The sample may be of any biological tissue or fluid. Frequently the sample will be a “clinical sample,” which is a sample derived from a patient. Such samples include, but are not limited to, blood, blood cells (e.g., white cells), tissue biopsy or fine needle aspiration biopsy samples, urine, peritoneal fluid, and pleural fluid, or cells therefrom. Biological samples may also include sections of tissues such as frozen sections taken for histological purposes.

The nucleic acid may be isolated from the sample according to any of a number of methods well known to those of skill in the art. One of skill in the art will appreciate that where expression levels of a gene or genes are to be detected, preferably RNA (mRNA) is isolated. Methods of isolating total mRNA are well known to those of skill in the art. For example, methods of isolation and purification of nucleic acids are described in Chapter 3 of Laboratory Techniques in Biochemistry and Molecular Biology (1993); Sambrook et al. (2001); Current Protocols in Molecular Biology (1987), all of which are incorporated herein by reference. Filter based methods for the isolation of mRNA are also known in the art. Examples of commercially available filter-based RNA isolation systems include RNAqueous® (Ambion) and RNeasy (Qiagen).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence. This provides an internal standard that may be used to calibrate the PCR reaction. The array may then include probes specific to the internal standard for quantification of the amplified nucleic acid.

Other suitable amplification methods include, but are not limited to polymerase chain reaction (PCR) (Innis, et al., 1990), ligase chain reaction (LCR) (see Wu and Wallace, 1989); Landegren, et al., 1988; Barringer, et al., 1990, transcription amplification (Kwoh, et al., 1989), and self-sustained sequence replication (Guatelli, et al., 1990).

In a particular embodiment, the sample mRNA is reverse transcribed with a reverse transcriptase, such as SuperScript II (Invitrogen), and a primer consisting of an oligo-dT and a sequence encoding the phage T7 promoter to generate first-strand cDNA. A second-strand DNA is polymerized in the presence of a DNA polymerase, DNA ligase, and RNase H. The resulting double-stranded cDNA may be blunt-ended using T4 DNA polymerase and purified by phenol/chloroform extraction. The double-stranded cDNA is then transcribed into cRNA. Methods for the in vitro transcription of RNA are known in the art and describe in, for example, Van Gelder, et al. (1990) and U.S. Pat. Nos. 5,545,522; 5,716,785; and 5,891,636, all of which are incorporated herein by reference.

If desired, a label may be incorporated into the cRNA when it is transcribed. Those of skill in the art are familiar with methods for labeling nucleic acids. For example, the cRNA may be transcribed in the presence of biotin-ribonucleotides. The BioArray High Yield RNA Transcript Labeling Kit (Enzo Diagnostics) is a commercially available kit for biotinylating cRNA.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense, as the target nucleic acids include both sense and antisense strands.

C. Labeling Nucleic Acids

To detect hybridization, it is advantageous to employ nucleic acids in combination with an appropriate detection means. Recognition moieties incorporated into primers, incorporated into the amplified product during amplification, or attached to probes are useful in the identification of nucleic acid molecules. A number of different labels may be used for this purpose including, but not limited to, fluorophores, chromophores, radiophores, enzymatic tags, antibodies, chemiluminescence, electroluminescence, and affinity labels. One of skill in the art will recognize that these and other labels can be used with success in this invention.

Examples of affinity labels include, but are not limited to the following: an antibody, an antibody fragment, a receptor protein, a hormone, biotin, Dinitrophenyl (DNP), or any polypeptide/protein molecule that binds to an affinity label.

Examples of enzyme tags include enzymes such as urease, alkaline phosphatase or peroxidase to mention a few. Colorimetric indicator substrates can be employed to provide a detection means visible to the human eye or spectrophotometrically, to identify specific hybridization with complementary nucleic acid-containing samples.

Examples of fluorophores include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluoroscein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

As mentioned above, a label may be incorporated into nucleic acid, e.g., cRNA, when it is transcribed. For example, the cRNA may be transcribed in the presence of biotin-ribonucleotides. The BioArray High Yield RNA Transcript Labeling Kit (Enzo Diagnostics) is a commercially available kit for biotinylating cRNA.

Means of detecting such labels are well known to those of skill in the art. For example, radiolabels may be detected using photographic film or scintillation counters. In other examples, fluorescent markers may be detected using a photodetector to detect emitted light. In still further examples, enzymatic labels are detected by providing the enzyme with a substrate and detecting the reaction product produced by the action of the enzyme on the substrate, and colorimetric labels are detected by simply visualizing the colored label.

So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin-bearing hybrid duplexes providing a label that is easily detected. For a detailed review of methods of labeling nucleic acids and detecting labeled hybridized nucleic acids see Laboratory Techniques in Biochemistry and Molecular Biology (1993).

D. Hybridization

As used herein, “hybridization,” “hybridizes,” or “capable of hybridizing” is understood to mean the forming of a double or triple stranded molecule or a molecule with partial double or triple stranded nature. The term “anneal” as used herein is synonymous with “hybridize.” The term “hybridization,” “hybridizes,” or “capable of hybridizing” are related to the term “stringent conditions” or “high stringency” and the terms “low stringency” or “low stringency conditions.”

As used herein “stringent conditions” or “high stringency” are those conditions that allow hybridization between or within one or more nucleic acid strands containing complementary sequences, but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as an mRNA or a nucleic acid segment thereof, or detecting at least one specific mRNA transcript or a nucleic acid segment thereof.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acids, the length and nucleobase content of the target sequences, the charge composition of the nucleic acids, and the presence or concentration of formamide, tetramethylammonium chloride or other solvents in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

The hybridization conditions selected will depend on the particular circumstances (depending, for example, on the G+C content, type of target nucleic acid, source of nucleic acid, and size of hybridization probe). Optimization of hybridization conditions for the particular application of interest is well known to those of skill in the art. Representative solid phase hybridization methods are disclosed in U.S. Pat. Nos. 5,843,663, 5,900,481, and 5,919,626. Other methods of hybridization that may be used in the practice of the present invention are disclosed in U.S. Pat. Nos. 5,849,481, 5,849,486, and 5,851,772.

1. DNA Chips and Microarrays

DNA arrays and gene chip technology provide a means of rapidly screening a large number of nucleic acid samples for their ability to hybridize to a variety of single stranded DNA probes immobilized on a solid substrate. These techniques involve quantitative methods for analyzing large numbers of genes rapidly and accurately. The technology capitalizes on the complementary binding properties of single stranded DNA to screen nucleic acid samples by hybridization (Pease et al., 1994; Fodor et al., 1991). Basically, a DNA array or gene chip consists of a solid substrate upon which an array of single stranded DNA molecules have been attached. For screening, the chip or array is contacted with a single stranded nucleic acid sample (e.g., cRNA), which is allowed to hybridize under stringent conditions. The chip or array is then scanned to determine which probes have hybridized.

The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly.

In general terms, hybridization is performed on an immobilized nucleic acid target or a probe molecule that is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules).

The Affymetrix GeneChip system may be used for hybridization and scanning of the probe arrays. In a preferred embodiment, the Affymetrix U133A array is used in conjunction with Microarray Suite 5.0 for data acquisition and preliminary analysis.

2. Normalization Controls

Normalization controls are oligonucleotide probes that are complementary to labeled reference oligonucleotides that are added to the nucleic acid sample. The signals obtained from the normalization controls after hybridization provide a control for variations in hybridization conditions, label intensity, “reading” efficiency and other factors that may cause the hybridization signal to vary between arrays. For example, signals read from all other probes in the array can be divided by the signal from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few normalization probes are used and they are selected such that they hybridize well (i.e. no secondary structure) and do not match any target-specific probes. Normalization probes can be localized at any position in the array or at multiple positions throughout the array to control for spatial variation in hybridization efficiently.

In a particular embodiment, a standard probe cocktail supplied by Affymetrix is added to the hybridization to control for hybridization efficiency when using Affymetrix Gene Chip arrays.

3. Expression Level Controls

Expression level controls are probes that hybridize specifically with constitutively expressed genes in the sample. The expression level controls can be used to evaluate the efficiency of cRNA preparation.

Virtually any constitutively expressed gene provides a suitable target for expression level controls. Typically expression level control probes have sequences complementary to subsequences of constitutively expressed “housekeeping genes.”

In one embodiment, the ratio of the signal obtained for a 3′ expression level control probe and a 5′ expression level control probe that specifically hybridize to a particular housekeeping gene is used as an indicator of the efficiency of cRNA preparation. A ratio of 1-3 indicates an acceptable preparation.

E. Data Analysis

Embodiments of the invention include methods to predict pathological response (pCR) versus residual cancer (RD) in patients diagnosed cancer prior to, during, or after treatment with a therapeutic regime. A variety of methods are know in the art for assessing the level of gene expression, as well algorithms to express these determinations as predictors, any combination of which may be used with the described gene set. In certain aspects, the prediction data may consist of baseline microarray gene expression data generated by hybridization of gene chips, e.g., U133A Affymetrix Gene Chips, consisting of 22,283 distinct probe sets corresponding to 13,736 known genes. This analysis is initiated by collecting various patient samples, which may include both pCRs and RDs. In certain embodiments, an array that has been hybridized with a population of nucleic acids isolated from a sample is scanned, images quantified, and preprocessed using the dCHIP© software or functionally similar software. The resulting data is assessed for quality (Gold, 2003a and 2003b).

Combining profiles of gene expression over a wide array of transcripts has potentially more classification prediction power than relying on any single gene. This contention relies implicitly on the intricate nature of gene-to-gene interactions and the host of possible molecular characteristics captured in genome wide RNA expression. Therefore, the issue addressed is which algorithm provides the better classifier, or combination thereof, to predict outcome given baseline gene expression. The search for a classifier involves spanning two spaces: classification algorithms and predictor sets (genes). Searching the space of all possible combinations of classifiers and gene sets is infeasible. Therefore, constraints may be imposed on the search spaces by: (1) limiting the choice of classification algorithms to a small discrete set and (2) searching over nested ordered subsets of genes, ordered by a measure of relative change in gene expression between outcomes.

Classifiers include, but are not limited to diagonal linear discriminant analysis (DLDA), support vector machines (SVM), compound co-variate predictor (CCP), and k-nearest neighbor algorithm (KNN), for K used in this context as the number of nearest neighbors (NN's) may be 3, 5, 7, 9, 11, or 15 (see Pusztai et al., 2003). The choices for the K# of NNs is selected based on previous CV simulations with public data that suggested that Ks in this range are reasonable. SVM was examined previously with publicly available microarray data (Mukherjee et al., 2003). DLDA and KNN were compared with various microarray data sets (Dudoit et al., 2000). CCP was examined with cancer microarray data (Tibshirani et al., 2002). The inventors choose to treat KNN for each K as a distinct model, although in actuality these are of adaptations of KNN, K being an internal parameter to KNN. These classifiers have been described in detail elsewhere (Hastie et al., 2001).

The inventors ordered the predictors, i.e. probe sets, considering nested sets. These were added based on an empirically derived order. The inventors ranked these with the p-value of a two-group, unequal variance, t-statistic on the ranks of gene expression. The inventors estimated validation prediction performance as the criteria for choosing between classifiers and employed Monte Carlo Cross Validation (MC-CV) to estimate of classification prediction performance.

Stratified K-Fold MC-CV entailed (i) dividing the sample data into an N-N/K training data set and an N/K test data set, each with roughly equal relative proportions of the two outcome classes, (ii) training each classifier on the training set, and (iii) obtaining prediction performance from the test set, and repeating r times. This is displayed in Algorithm 1. The choice of K, not to be confused with the K# of NNs, is addressed below.

Algorithm 1 for stratified K-fold MC-CV includes (1) Divide data into an N-N/K sample training data set and a N/K sample test set, each with roughly equal relative proportions of each class; (2) Train model on training data set; (3) Measure and record prediction performance applying model to test data set; (4) Repeat steps 1-3 a total of r times; and (5) Summarize resulting r performance measures.

One of the preliminary questions was whether feature, or gene, selection should be an integral part of the MC-CV. Feature selection is discussed in more detail below. The inventors also examined how many MC-CV repetitions, r, to do. The inventors chose as a starting value r=100, with the rationale that the variation in the mean of a proportion summarizing performance would be little reduced beyond this point. However, the inventors further evaluated this choice beyond just mean performance. Choosing r the number of MC-CV iterations is discussed in more detail below.

The inventors also considered how to best choose K. Additionally, various methods for choosing a best classifier(s) and a gene set from the candidates were considered. For each MC-CV run the inventors recorded: accuracy (ACC), true positive fraction (TPF) or sensitivity, false positive fraction (FPF) or 1-specificity, positive predictive value (PPV) and negative predictive value (NPV) (Pepe et al., 2003). The inventors also recorded sample level performance to determine which samples were the most troublesome. In certain embodiments, the analysis was focused on ACC.

1. Choosing K for K-Fold CV.

Initially, feature selection was not incorporated with CV. The genes were ranked using all training samples and included to form ever-larger nested predictor sets. The inventors considered K=10-fold cross validation (Leave-6-Out), K=2-fold cross validation (Leave-30-Out) and K=N (Leave-1-Out) Kohvai, 1995; Shoa, 1993). The inventors inspected these results to learn how much the mean ACC and the confidence interval of the ACC changed as a function of K. With K=10, K=2, and K=N, that for each classifier shown, the mean test ACC does not change dramatically with K. However, the spread in the confidence intervals of the ACC decreases substantially from the Leave-6-Out test to the Leave-30-Out test, indicating that the estimates are much more precise with Leave-30-Out cross validation.

Given that the spread was dramatically reduced for K=2 while at the same time mean ACC declined only slightly the inventors chose to use K=2 for subsequent MC-CV studies. Note, however, that this will only result in prediction performance for training set sizes of N/2. The inventors do not expect steep learning curves and therefore, this is considered reasonable.

2. Feature (Gene) Selection.

A BUM (Pounds and Morris, 2003) analysis using all samples resulted in an appreciable number of genes showing change between outcomes. There were 19 selected for a false discovery rate (FDR) of 1% and 150 for a FDR of 5%.

In certain embodiments, feature selection is included within MC-CV iterations, as described above, and may result in a more honest assessment of the prediction performance. This would entail for every split of the data into training and test set, re-ranking the genes based on the training data alone. Repeating the gene ranking each time does entail use of more CPU time and one time saver is to use the same r random samples in order to divide the data into training and test sets for each classifier/gene set. The main computing advantage is that one only needs to derive the ranks for each split once, store and access them over r iterations. An additional advantage is the reduction in confounding between the subsampling and factors for comparison. Hence, the rankings for r=100 random training sets were computed ahead of time and stored up front for use later.

The inventors examined the variability in ranks for leave out sets using MC-CV. There was much more variability past the top 100 genes. As the number of training samples increases, the inventors would hope for this variability to decline. Although, the feature selection using just a fraction of the 22,283 genes, for KNN (K=5), may produce overly optimistic results. Here the mean ACC's without feature selection or feature selection from just a subset of 1000 of the top genes are higher by as much as 5% in some ranges depending on the number of genes in the model.

In conclusion, incorporation of feature selection in MC-CV is an important devise for helping to better access achievable prediction performance. Feature selection preferably conducted using all 22,283 genes, rather than a subset, as the empirical evidence shows that this can make a difference in the end results and final decision.

3. Choosing r the Number of MC-CV Iterations.

For several classifiers the mean and standard error estimates for 2-fold MC-CV with feature selection of 20 genes for up to r=300 show that convergence of the sample means is reasonable after r=100 repetitions, although the standard errors do not begin to calm until after 200 repetitions. The inventors selected r that would allow a sample of sufficient size to estimate the ACC sample mean and standard error, while saving the extra computing time that would be required for more repetitions. Moreover, this would reduce the standard error in the mean ACC estimate to a level that was sufficiently low.

4. Choosing the Classifier.

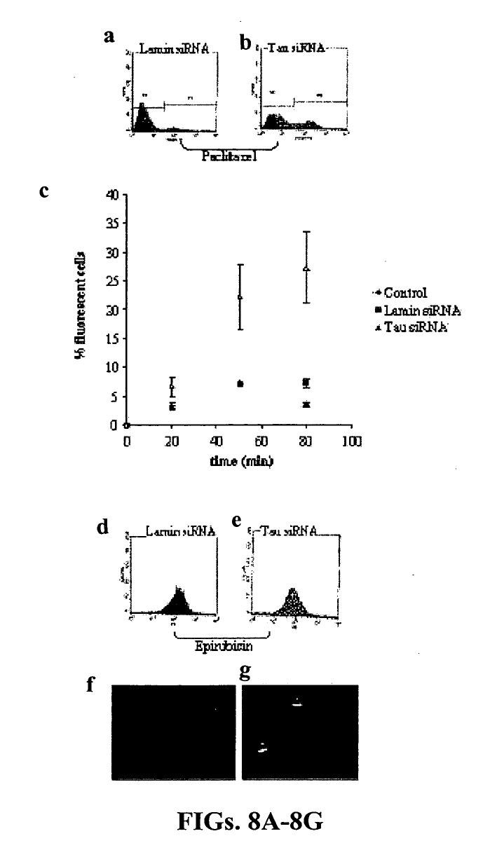

To define the best predictors, the inventors postulated that classifiers with mean ACCs within 1 standard deviation of the single best should also be considered as best candidates. In 2-fold MC-CV with feature selection, KNN k=7 achieved the highest accuracy of 76% at 20 genes with a 1 standard error lower bound of 69% (FIG. 8). Each of the other KNN classifiers achieved above this lower bound as well as SVM with greater than 15 genes. Any of these predictors can be considered as a good candidate for best predictor.

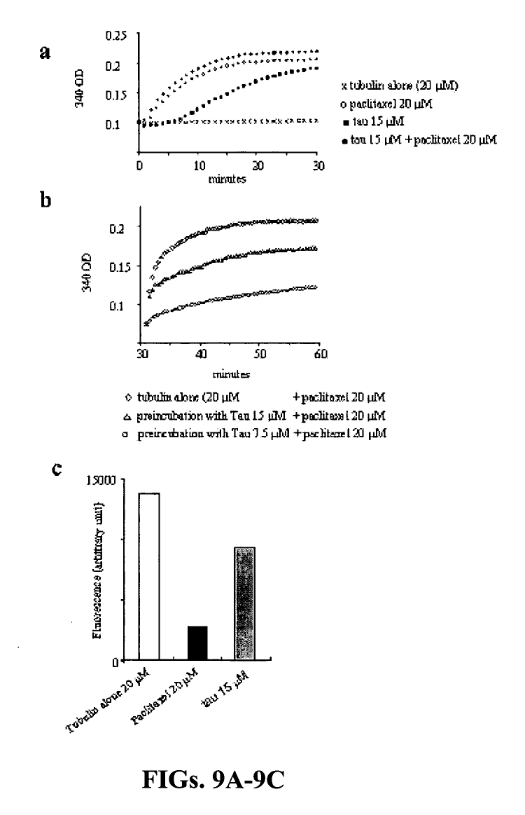

Without including feature selection in the models, SVM achieved the best ACC of 89% with 125 genes and standard error of 0.04 (FIG. 9). However, those results are conditional upon the gene ranks using all 60 samples to perform the t-tests, ranks that are a random sample from a larger population. K-NN k=7 with 20 genes showed ACC of 78%. What these show is that KNN K=7 stands up as more robust in the face of uncertainty due to feature selection, for training sample sizes of 30, than SVM. The preferred final classifier was k-NN k=7 because it is predicted to perform better in achieving high ACC in the face of not only uncertainty in validation prediction (estimated with MC-CV) but also with the feature selection (estimated with MC-CV including feature selection) as well.

5. Permutation Testing of the Best Classifier

Permutation testing of classification accuracy (ACC) is a powerful method to assess whether or not the accuracy that is achieved in a given study was significant (Mukherjee et al., 2003). The method begins with Algorithm 1 followed by permutation of class labels (i.e. response outcome), repeating Algorithm 1 Q times and comparing the original accuracy with those obtained via permutation, ACCqPERM q=1, . . . , Q.

Typically, the comparison is achieved by calculating the percentage of cases for which ACC is greater than or equal to ACCPERM. This measure is taken to be an empirical estimate of the p-value. For large Q it can be shown that in many situations this method is unbiased and robust against alternatives that do not take into account the underlying unique structure of the data (Good, 1994).

Permutation testing of ACC using Algorithm 2 includes (1) Perform Algorithm 1 and summarize ACC; (2) Randomly permute the class labels; (3) Repeat Algorithm 1, recording ACC^{PERM} at each run; (4) Repeat steps 2-3 Q times; and (5) Summarize comparison of ACC with ACC^{PERM} obtained by permuting the labels.

Significance in this case is a measure of whether or not the ACC achieved was better than chance, e.g. the permutation test. In the case of two groups with balance, i.e. the number of replicates in both groups equal, the null hypothesis with the permutation testing is defined as Ho: ACCTRUE=50% versus the alternative that Ha: ACCTRUE>50%. Hence, ACC arbitrarily close to 50% may be rejected as significant with enough samples, i.e. power, although ACC this low is rarely practical in medical decision making.

II. Isolated Nucleic Acids for Analysis or Therapy

Nucleic acids of the present may be utilized in the preparation of therapeutic compositions. Certain genes related to the sensitivity of a cell to therapy that are expressed in a cell sensitive to therapy may be used therapeutically by increasing the expression of this gene or activity of an encoded protein in a cancer cell. Other genes related to resistance of a cell to a therapy may be down regulated transcriptionally or inhibited at the protein level by various therapies, such as anti-sense nucleic acid methods or small molecules. The protein products of these genes may also be targets for small molecules and the like, to either increase activity of a sensitizing protein or decrease activity of a resistance protein. Therapeutics that target the transcription of a gene, translation of RNA, and/or activity of an encoded protein may be used to sensitize cells to therapy, or in other aspects, may be used as a primary therapeutic apart from or in combinations with other therapies.

Nucleic acids of the present invention include nucleic acid isolated from a sample, probes, or expression vectors for both analysis of tumor responsiveness to therapy and cancer therapy. Certain embodiments of the present invention include the evaluation of the expression of one or more nucleic acids of SEQ ID NOS: 1-193. In certain embodiments, wild-type, variants, or both wild-type and variants of these sequences are employed. In particular aspects, a nucleic acid encodes for or comprises a transcribed nucleic acid. In other aspects, a nucleic acid comprises a nucleic acid segment of one or more of SEQ ID NOS: 1-193, or a biologically functional equivalent thereof.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). “Nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

In, certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. The term “gene” includes both genomic sequences, RNA or cDNA sequences or smaller engineered nucleic acid segments, including non-transcribed nucleic acid segments, including but not limited to the non-transcribed promoter or enhancer regions of a gene. Smaller engineered nucleic acid segments may encode proteins, polypeptides, peptides, fusion proteins, mutants and the like.

A polynucleotide of the invention may form an “expression cassette.” An “expression cassette” is polynucleotide that provides for the expression of a particular transcription unit. A transcription unit may include promoter elements and various other elements that function in the transcription of a gene or transcription unit, such as a polynucleotide encoding all or part of a therapeutic protein. An expression cassette may also be part of a larger replicating polynucleotide or expression vector.

“Isolated substantially away from other coding sequences” means that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

A. Expression Constructs

Expression constructs of the invention may include nucleic acids encoding a protein or polynucleotide for use in cancer therapy. In certain embodiments, genetic material may be manipulated to produce expression cassettes and expression constructs that encode the nucleic acids or inhibitors of the nucleic acids of the invention. Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of therapeutic genes.

A therapeutic vector of the invention comprises a therapeutic gene for the prophylatic or therapeutic treatment of neoplastic, hyperplastic, or cancerous condition. In order to mediate the expression of a therapeutic gene in a cell, it will be necessary to transfer the therapeutic expression constructs into a cell. Such transfer may employ viral or non-viral methods of gene transfer. Gene transfer may be accomplished using a variety of techniques known in the art, including but not limited to adenovirus, various retroviruses, adeno-associated virus, vaccinia virus, canary pox virus, herpes viruses or other non-viral methods of nucleic acid delivery.

Various methods and compositions for nucleic acid transfer, both ex vivo and in vivo may be found in the following references: Carter and Flotte, 1996; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996; McCown et al., 1996; Ping et al., 1996; Ridgeway, 1988; Baichwal and Sugden, 1986; Coupar et al., 1988. Other methods of gene transfer include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), naked DNA expression construct (Klein et al., 1987; Yang et al., 1990), Liposomes (Ghosh and Bachhawat, 1991; Radler et al., 1997; Nicolau et al. 1987; Kaneda et al., 1989; Kato et al., 1991) and receptor-mediated transfection (Wu and Wu, 1987; Wu and Wu, 1988).

1. Control Regions

Expression cassettes or constructs of the invention, encoding a therapeutic gene will typically include various control regions. These control regions typically modulate the expression of the gene of interest. Control regions include promoters, enhancers, polyadenylation signals, and translation terminators. A “promoter” refers to a DNA sequence recognized by the machinery of the cell, or introduced machinery, required to initiate the specific transcription of a gene. In particular aspects, transcription may be constitutive, inducible, and/or repressible. The phrase “under transcriptional control” means that the promoter is in the correct location and orientation in relation to the nucleic acid to control RNA polymerase initiation and expression of the gene.

In various embodiments, the human cytomegalovirus immediate early gene promoter (CMVIE), the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral, retroviral or mammalian cellular or bacterial phage promoters, which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic. For example, the ecdysone system (Invitrogen, Carlsbad, Calif.) and Tet-Off™ or Tet-On™ system (Clontech, Palo Alto, Calif.) are two such systems.

In some circumstances, it may be desirable to regulate expression of a transgene in a therapeutic expression vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues.

Tumor specific promoters such as osteocalcin, hypoxia-responsive element (HRE), MAGE-4, CEA, alpha-fetoprotein, GRP78/BiP and tyrosinase may also be used to regulate gene expression in tumor cells.

It is envisioned that any of the above promoters alone or in combination with another may be useful according to the present invention depending on the action desired. In addition, this list of promoters should not be construed to be exhaustive or limiting, those of skill in the art will know of other promoters that may be used in conjunction with the promoters and methods disclosed herein.

Enhancers may also be utilized in construction of an expression vector. Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Polyadenylation signals may be used in therapeutic expression vectors. Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

B. Therapeutic Genes

Genes identified as either sensitizing genes or resistance genes may be targeted for therapeutic expression or repression, respectively. The present invention contemplates the use of a variety of different therapeutic genes. For example, genes encoding enzymes, hormones, cytokines, oncogenes, receptors, ion channels, tumor suppressors, transcription factors, drug selectable markers, toxins, various antigens, anti-sense polyunucleotide and other inhibitors of gene expression are contemplated for use according to the present invention. In certain embodiments, a therapeutic gene may encode an anti-sense polynucleotide, siRNA, or ribozymes that interfere with the function of DNA and/or RNA. Interference may result in suppression of expression, in particular aspects expression of Tau protein. The presence or expression of such a polynucleotide or derivative thereof in a cell will typically alter the expression or function of cellular genes or RNA.

C. Multigene Constructs and IRES

In certain embodiments of the invention, the use of internal ribosome binding sites (IRES) elements are used to create multigene, polycistronic messages. IRES elements are able to bypass the ribosome scanning model of 5′-methylated, Cap-dependent translation and begin translation at internal sites (Pelletier and Sonenberg, 1988). RES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple genes can be efficiently expressed using a single promoter/enhancer to transcribe a single message. Any heterologous open reading frame can be linked to IRES elements. This includes genes for therapeutic proteins and selectable markers. In this way, expression of several proteins can be simultaneously engineered into a cell with a single construct and a single selectable marker.

D. Preparation of Nucleic Acids

In addition to the preparation of nucleic acids from a tumor sample and isolated nucleic acid may be prepared as follows. An isolated nucleic acid may be made by any technique known to one of ordinary skill in the art, such as for example, chemical synthesis, enzymatic production, or biological production. Non-limiting examples of a synthetic nucleic acid (e.g., a synthetic oligonucleotide), include a nucleic acid made by in vitro chemical synthesis using phosphotriester, phosphite, or phosphoramidite chemistry; and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

E. Purification of Nucleic Acids

A nucleic acid may be purified on polyacrylamide gels, cesium chloride centrifugation gradients, affinity columns, or by any other means known to one of ordinary skill in the art (see for example, Sambrook et al., 2001, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

1. Nucleic Acid Segments

In certain embodiments, the nucleic acid is a nucleic acid segment. As used herein, the term “nucleic acid segment,” are smaller fragments of a nucleic acid, such as those that encode only part of the SEQ ID NOS: 1-193. Thus, a “nucleic acid segment” may comprise any part of a gene sequence, from about 8 nucleotides to the full length of the SEQ ID NOS: 1-193.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm would be applied to each of SEQ ID NOS: 1-193. As used herein, a “probe” generally refers to a nucleic acid used in a detection method or composition. As used herein, a “primer” generally refers to a nucleic acid used in an extension or amplification method or composition.

In a non-limiting example, one or more nucleic acid constructs may be prepared that include a contiguous stretch of nucleotides identical to or complementary to one or more of SEQ ID NOS: 1-193. A nucleic acid construct may be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 60, about 70, about 80, about 90, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

III. Pharmaceutical Compositions and Routes of Administration

Where clinical applications are contemplated, it will be necessary to prepare pharmaceutical compositions of the therapeutic compositions in a form appropriate for the intended application. Generally, this will entail preparing compositions that are essentially free of pyrogens, as well as other impurities that could be harmful to humans or animals.

One will generally desire to employ appropriate salts and buffers to render the compositions suitable for introduction into a patient. Aqueous compositions of the present invention comprise an effective amount of the gene delivery agent dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances for gene delivery agents are well know in the art. Except insofar as any conventional media or agent is incompatible with the vectors or cells of the present invention, its use in therapeutic compositions is contemplated.

An effective amount of the composition is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

Also contemplated are combination compositions that contain two active ingredients. In particular, the present invention provides for compositions that contain expression vector compositions and at least a second therapeutic, for example, an anti-neoplastic drug.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media can be employed and is known to those of skill in the art. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of Responsiveness Genes and Development of Multi-Gene Predictors of Response to Chemotherapy

Methods

The inventors have identified a set of 193 genes that are differentially expressed between breast cancers that are highly chemotherapy sensitive and those which are less sensitive. These genes were identified by comprehensive gene expression profiling using Affymetrix U133A and B gene chips on fine needle aspiration specimens of at least 85 human breast cancers obtained at the time of diagnosis, before therapy. All patients received sequential weekly paclitaxel (P)×12 followed by 4 additional courses of 5-FU, doxorubicine, and cyclophosphamide (FAC) preoperative chemotherapy. These 193 genes, including subsets of these genes, combined with a prediction algorithm can be used to identify patients at the time of diagnosis who have better than average probability to experience complete eradication of the cancer (pathologic complete response, pCR) to P/FAC chemotherapy.

Patient Population. All patients were enrolled in a clinical trial at M.D. Anderson Cancer Center (LAB99-402). Patients were grouped into two groups based on pathologic response outcome determined by pathologic examination of the surgically resected breast tissues after completion of six months of chemotherapy. Twenty-one of 82 patients had pathologic complete response (pCR) and 61 of 82 patients had residual disease (RD). The chemotherapy consisted of weekly paclitaxel 80 mg/m²×12 courses followed by four additional treatments with a combination of 5-fluorouracil (500 mg/m²), doxorubicin (50 mg/m²) 72-hour infusion, and cyclophosphamide (500 mg/m²) given once every 3 weeks. All patients received 24 weeks of sequential T/FAC chemotherapy and subsequently underwent lumpectomy or modified radical mastectomy with axillary node sampling as determined appropriate by the surgeon. Metallic markers had been placed under radiological guidance in the shrinking tumor bed for any patient whose tumor became <1 cm by imaging during the course of treatment. Clinical characteristics and treatment history are presented in Table 2. At the completion of neoadjuvant chemotherapy all patients had surgical resection of the tumor bed, with negative margins. Grossly visible residual cancer was measured and representative sections were submitted for histopathologic study. When there was not grossly visible residual cancer, the slices of the specimen were radiographed and all areas of radiologically and/or architecturally abnormal tissue were entirely submitted for histopathologic study. This study was approved by the institutional review board (IRB) of MDACC and all patients signed an informed consent for voluntary participation.

Fine Needle Aspiration. Fine needle aspiration (FNA) was performed using a 23 or 25-gauge aspiration needle (local anesthesia with ethyl chloride spray). Four to six FNA passes were obtained and two passes of each placed into separate vials containing 0.5 ml RNAlater™ solution (Ambion, Austin, Tex.) and mixed thoroughly. The samples in RNAlater™ solution were kept at room temperature for 20-30 minutes then snap frozen and stored at −80° C.

One cytologic smear was prepared from the last FNA by placing a drop of cellular material on a silane-coated slide and air-dried. The adequacy and cellularity of the sample was assessed by examining the DiffQuik (Baxter Scientific, Illinois, U.S.A)-stained cytologic smear under the microscope. Typically, an FNA specimen contains 78-90% neoplastic cells, few infiltrating leukocytes and few red cells. These samples contain little or no stromal cells (fibroblast, adipocyes) or normal breast epithelium.

RNA Extraction. The Qiagen Rneasy Mini Kit Cat # 74104 was used for RNA extraction from the FNA samples that were stored in RNAlater™ solution at −80° C. The samples were thawed on ice and then spun in a 5415C eppendorf centrifuge at 10,000 rpm for 5 minutes.

As much of the supernatant as possible (approx. 900 ul) was carefully removed and transfered to a new 1.5 ml eppendorf tube labeled with the patient ID. The supernatant was stored at −80° C. for future processing as it is possible to get RNA from the supernatant.

TABLE 2
Clinical information and demographics of the patients included in the
study (n = 82)

| | |
|---|---|
| Female | 82 (100%) |
| Median age | 52 years (range 29-79) |
| Race | |
| Caucasian | 56 (68%) |
| African American | 11 (13%) |
| Asian | 7 (9%) |
| Hispanic | 6 (7%) |
| Mixed | 2 (2%) |
| Histology | |
| Invasive ductal | 73 (89%) |
| Mixed ductal/lobular | 6 (7%) |
| Invasive lobular | 1 (1%) |
| Invasive mucinous | 2 (2%) |
| TNM stage | |
| T1 | 7 (9%) |
| T2 | 46 (56%) |
| T3 | 15 (18%) |
| T4 | 14 (17%) |
| N0 | 28 (34%) |
| N1 | 38 (46%) |
| N2 | 8 (10%) |
| N3 | 8 (10%) |
| Nuclear grade (BMN) | |
| 1 | 2 (2%) |
| 2 | 23 (37%) |
| 3 | 35 (61%) |
| ER positive¹ | 35 (43%) |
| ER negative | 47 (57%) |
| HER-2 positive² | 57 (70%) |
| HER-2 negative | 25 (30%) |
| Neoadjuvant therapy³ | |
| Weekly T (80 mg/m²) × 12 + FAC × 4 | 69 (84%) |
| 3-weekly T (225 mg/m CI) × 4 + FAC × 4 | 13 (16%) |
| Pathologic complete response (pCR) | 21 (26%) |
| Residual Disease (RD) | 61 (74%) |

¹Cases where ≧10% of tumor cells stained positive for ER with immunohistochemistry (IHC) were considered positive.
²Cases that showed either 3 + IHC staining or had gene copy number >2.0 were considered HER-2 “positive”.
³T stands for paclitaxel, FAC for 5-flurouracil, doxorubicin, and cyclophosphamide.

Next, 350 μl of RLT lysis buffer (Qiagen) was added to the cell pellet and mixed thoroughly by pippetting and vortexing. A quick spin down in the 5415C centrifuge at 14,000 rpm was performed, and the cells were transferred to a new 0.5 ml eppendorf tube labeled with the appropriate patient ID.

The cells were homogenized by passing through a 30.5G needle with a 1 ml syringe 10-20 times. After homogenization, the samples were vortexed and spun down. The homogenized sample was then transferred to a new 1.5 ml eppendorf tube labeled with the appropriate patient ID. Next, 350 ul of 70% ethanol solution was added to the sample and mixed by pippettintg.

Then 700 μl of the sample was applied to an RNeasy® mini column placed in a 2 ml collection tube. The tube was placed in the 5415C eppendorf centrifuge and spun at 14,000 rpm for 15 seconds. The flow through was discarded. 700 μl of buffer RW1 was then added to the RNeasy® column. The tube was centrifuged in the 5415C for 15 seconds at 14,000 rpm, and the flow through was discarded.

The RNeasy® column was transferred to a new 2 ml collection tube. 500 μl of Buffer RPE was pipetted onto the column. The tube was centrifuged in the 5415C for 15 seconds at 14,000 rpm to wash the column. The flow through was discarded.

The RNeasy® column was then transferred to another new 2 ml collection tube. 500 ul of Buffer RPE was pipetted onto the column. The tube was centrifuge in the 5415C for 2 minutes at 14,000 rpm. The flow through was discarded.

The RNeasy® mini column was then transferred to a 1.5 ml eppendorf tube. 40 μl of RNase free water was pipetted onto the middle of the silica membrane. The tube was spun in the 5415C centrifuge for 1 minute at 14,000 rpm to elute the RNA. The 40 μl elution was transferred back onto the RNeasy® mini column and spun for a second time in the 5415C centrifuge for 1 minute at 14,000 rpm.

The 40 μl volume sample was then concentrated in a Sorvall speed-vac to a final volume of 10-15 μl.

To determine the amount of RNA in the sample, a 1:50 dilution of the sample was diluted in a total volume of 50 μl in a miniature cuvette (Beckman), and the amount and quality of RNA was assessed with DU-640 U.V. Spectrophotometer (Beckman Coulter, Fullerton, Calif.). It was considered adequate for further analysis if the OD 260/280 ratio was >1.8 and the total RNA yield was >1 μg. Median RNA yield of the 85 specimens was 2.0 μg with a range of 1 μg-22 μg. Between 0.9 μg to 1.1 μg total RNA in a 9 μl volume was used for Affymetrix Labeling.

Affymetrix Probe Preparations and Hybridization. All procedures followed standard operating practice described in the Affymetrix technical manual. Briefly, total RNA was reverse-transcribed with SuperScript II in the presence of T7-(dT)₂₄ primer to generate first strand cDNA. A second-strand cDNA synthesis was performed in the presence of DNA Polymerase I, DNA ligase, and RNase H. The resulting double-stranded cDNA was blunt-ended using T4 DNA polymerase and purified by phenol/chloroform extraction. This double-stranded cDNA was transcribed into cRNA in the presence of biotin-ribonucleotides using the BioArray High Yield RNA transcript labeling kit (Enzo Laboratories). The biotin labeled cRNA was purified using Qiagen RNeasy columns and quantified. A minimum of 10 μg cRNA is required in order to proceed with fragmentation and hybridization.

cRNA was fragmented at 94° C. for 35 minutes in the presence of 1× fragmentation buffer and then hybridized to Affymetrix U133A arrays overnight at 42° C. After hybridization, cRNA was recovered from the chips and stored at −80° C. The Affymetrix GeneChip system was used for hybridization and scanning of the probe arrays. Microarray Suite 5.0 was used for data acquisition and preliminary analysis. Grid alignment was checked by plotting the signal of positive and negative controls versus border position and the pixel-level coefficient of variation within each cell. Primary data was normalized to the median of each chip by setting the median value to 1000 and log 2 transformed for further analysis.

QC process for cRNA labeling and hybridization. To control for hybridization efficiency a standard probe cocktail supplied by Affymetrix was spiked into the hybridization mix. After hybridization and staining of the chip, the signal analysis software checks for successful hybridization present at the cells corresponding to the spiked-in cRNA. The expression of known housekeeping genes represented on the chip was also examined to evaluate the efficiency of cRNA preparation. For housekeeping genes on the chip a ratio of the signal obtained for 3′ and 5′ probes was used as an indicator of the efficiency of cRNA preparation. A ratio of 1-3 indicates an acceptable preparation of cRNA. Several standard global quality metrics were also examined to further assure good quality data. To assess brightness, dCHIP software was used to generate % of array-outliers and % single-outliers for each chip. Affymetrix MAS 5.0 software was used to produce p-values for signal detection. These were compared to all the rest of the existing profiles. Chips with greater than 5% array- or single-outliers or with less than 15% detection p-values of <0.01 were flagged and discarded from further analysis. The median of the median intensities over all the arrays was 163 with a range of 228 and a standard deviation of 42.9. Three chips failed the QC process and subsequent analysis was performed on 82 samples.

Microarray data analysis. The inventors' goal was to predict pathological response (pCR) versus residual cancer (RD) in patients with newly diagnosed breast cancer following neoadjuvant therapy. The prediction data consisted of baseline microarray gene expressions generated by U133A Affymetrix Gene Chips, consisting of 22,283 distinct probe sets, i.e. distinct target sequences, corresponding to 13,736 known genes. This analysis was based on 82 patient samples, 21 pCRs and 61 RDs. The scanned images were quantified and then preprocessed using the dCHIP© software. The resulting data was assessed for quality. Data preprocessing and quality control were discussed previously (Gold, 2003a and 2003b). dCHIP software was used for normalization; this program normalizes all arrays to one standard array that represents a chip with median overall intensity. After normalization, probe set level intensity estimates were generated as follows. Estimates of feature level intensity was derived from the 75th percentile of each features' pixel level intensities. Each individual probe is aggregated at the feature level to form a single measure of intensity for each probe set. The inventors used the perfect match model. Normalized gene expression values were transformed to the log-scale (base 10) for analysis. To identify informative genes differentially expressed between cases with pCR and those with residual disease, genes were ordered by p-values obtained with two-sample, unequal-variance t-tests.

Combining profiles of gene expression over a wide array of transcripts has potentially more classification prediction power than relying on any single gene. This contention relies implicitly on the intricate nature of gene-to-gene interactions and the host of possible molecular characteristics captured in genome wide RNA expression. Therefore, the issue addressed here is which algorithm provides the better classifier, or combination thereof, to predict outcome given baseline gene expression. The search for a classifier involved spanning two spaces: classification algorithms and predictor sets (genes). Searching the space of all possible combinations of classifiers and gene sets is infeasible. Therefore, constraints were imposed on the search spaces by: (1) limiting the choice of classification algorithms to a small discrete set and (2) searching over nested ordered subsets of genes, ordered by a measure of relative change in gene expression between outcomes.

Multigene classifiers were constructed using combinations of the most informative genes and several different class prediction algorithms including Support Vector Machines with linear, radial and polynomial kernels (SVM), Diagonal Linear Discriminant Analysis (DLDA), and K-Nearest Neighbor (KNN) using Euclidean distance (Hastie et al., 2001). Monte Carlo Cross Validation (CV) was used to estimate the prediction performance of the different classifiers in the training data and to facilitate selection of a final single best classifier for independent validation. Use of cross-validation avoids the optimism bias that occurs when the same data are used to assess the performance of a classifier and to train the classifier. The inventors examined the DLDA, SVM, CCP, and KNN, for K used in this context as the number of nearest neighbors (NN's) of 3, 5, 7, 9, 11, 15 classifiers. The choices for the K# of NNs was selected based on previous CV simulations with public data that suggested that Ks in this range are reasonable. SVM was examined previously with publicly available microarray data (Mukherjee et al., 2003). DLDA and KNN were compared with various microarray data sets (Dudoit et al., 2000). CCP was examined with cancer microarray data (Tibshirani et al., 2002). The inventors choose to treat KNN for each K as a distinct model, although in actuality these are of adaptations of KNN, K being an internal parameter to KNN. These classifiers have been described in detail elsewhere (Hastie et al., 2001).

The inventors ordered the predictors, i.e. probe sets, considering nested sets. These were added based on an empirically derived order. The inventors ranked these with the p-value of a two-group, unequal variance, t-statistic on the ranks of gene expression. The inventors estimated validation prediction performance as the criteria for choosing between classifiers and employed Monte Carlo Cross Validation (MC-CV) to estimate of classification prediction performance.

Stratified K-Fold MC-CV entailed (i) dividing the N=82 sample data into an N-N/K training data set and an N/K test data set, each with roughly equal relative proportions of the two outcome classes, (ii) training each classifier on the training set, and (iii) obtaining prediction performance from the test set, and repeating r times. This is displayed in Algorithm 1. The choice of K, not to be confused with the K# of NNs, is addressed below.

Algorithm 1 for stratified K-fold MC-CV includes (1) Divide data into an N-N/K sample training data set and a N/K sample test set, each with roughly equal relative proportions of each class; (2) Train model on training data set; (3) Measure and record prediction performance applying model to test data set; (4) Repeat steps 1-3 a total of r times; and (5) Summarize resulting r performance measures.

One of the preliminary questions was whether feature, or gene, selection should be an integral part of the MC-CV. Feature selection is discussed in more detail below. The inventors also examined how many MC-CV repetitions, r, to do. The inventors chose as a starting value r=100, with the rationale that the variation in the mean of a proportion summarizing performance would be little reduced beyond this point. However, the inventors further evaluated this choice beyond just mean performance. Choosing r the number of MC-CV iterations is discussed in more detail below.

The inventors also considered how to best choose K. Additionally, various methods for choosing a best classifier(s) and a gene set from the candidates were considered. For each MC-CV run the inventros recorded: accuracy (ACC), true positive fraction (TPF) or sensitivity, false positive fraction (FPF) or 1-specificity, positive predictive value (PPV) and negative predictive value (NPV) (Pepe et al., 2003). The inventors also recorded sample level performance to determine which samples were the most troublesome. The inventors focused their analysis here on ACC. Choosing the best classifier is discussed in more detail below.

Choosing K for K-fold CV. Cross validation was performed by repeated iteration (n=100) of stratified random sampling from a full data set to estimate expected performance for independent test cases. Stratification was performed to insure that the relative proportion of outcomes sampled in both cross-validation training and test sets was similar to the original proportions for the full training data. Gene sorting was included in the cross-validation to avoid selection bias (Ambroise and McLauchlan, 2002). The inventors performed 2-, 4-, 10-, 20-, and 40-fold CV but focus on 2-fold because it has lower variation in the performance estimates over the 100 iterations and this lower variation facilitates choosing among the competing classifiers. Classifier performance was assessed using overall misclassification error (MER), which is the proportion of samples misclassified and by using the complement of the area under the Receive Operator Characteristic curve (or area above the curve, AAC). The latter is generally considered a superior measure of performance because it offers a balance between sensitivity and specificity and is not dependent on the class proportions in the way that overall accuracy is (Pepe, 2003). Random label permutation testing was used to assess whether the performance achieved with our chosen classifier was significant (Hsing et al., 2003).

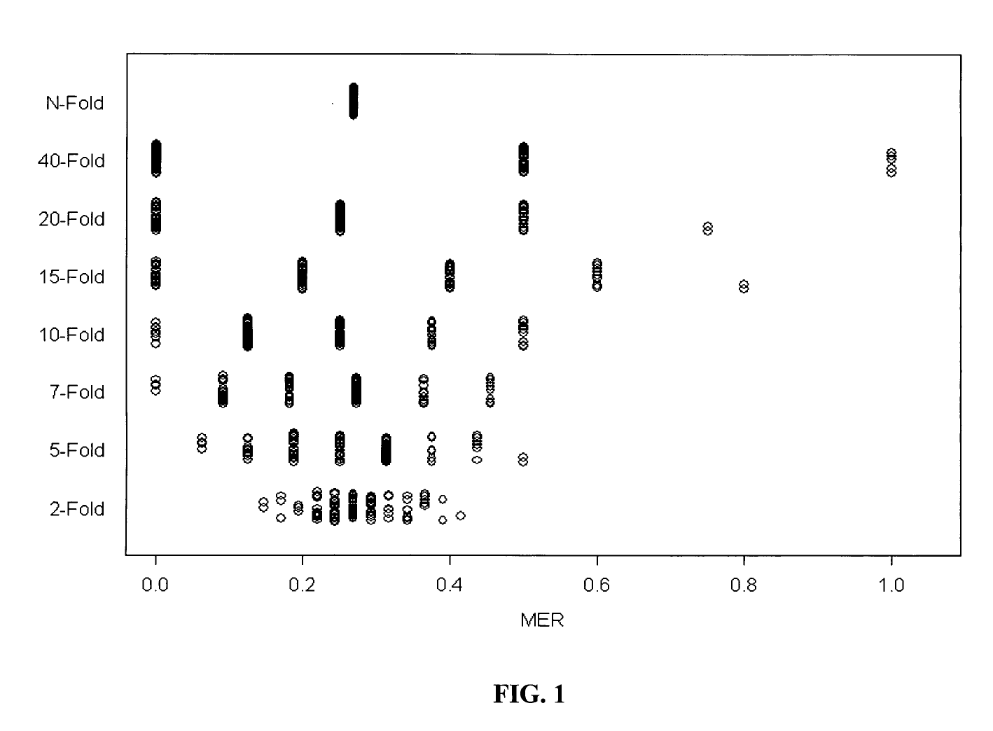

Cross-validation. FIG. 1 is a dot plot of the fully cross-validated misclassification results for a particular classifier (DLDA with 30 genes) over the 100 iterations for 2-, 5-, 7-, 10-, 15-, 20-, 40- and 82-fold cross-validation. Leave-one-out cross-validation is equivalent to 82-fold cross-validation when there are 82 samples. As the number of folds increases, the number of test samples decreases, e.g., with 2-fold CV, the inventors test on 41 samples, with 10-fold CV the inventors test on about 8 samples, and with 40-fold CV the inventors test on 2 samples. The decrease in the number of test samples has at least two consequences. First, it increases the discreteness of the results, e.g., with the 40-fold CV using 2 test samples, there are only three possible values for the misclassification error (0/2, 1/2, or 2/2). The second consequence is an increase in the variation of the results, the SD is 6% for 2-fold, 10% for 5-fold, 14% for 10-fold, 19% for 20-fold, and 30% for 40-fold. Based on these and similar results for other measures of performance, the inventors chose to focus attention on the 2-fold CV results.

Permutation Testing of the Best Classifier (K-NN, k=7, 20-gene). Permutation testing of classification accuracy (ACC) is a powerful method to assess whether or not the accuracy that is achieved in a given study was significant (Mukherjee et al., 2003). The method begins with Algorithm 1 followed by permutation of class labels (i.e. response outcome), repeating Algorithm 1 Q times and comparing the original accuracy with those obtained via permutation, ACCqPERM q=1, . . . , Q.

Typically, the comparison is achieved by calculating the percentage of cases for which ACC is greater than or equal to ACCPERM. This measure is taken to be an empirical estimate of the p-value. For large Q it can be shown that in many situations this method is unbiased and robust against alternatives that do not take into account the underlying unique structure of the data (Good, 1994).

Permutation testing of ACC using Algorithm 2 includes (1) Perform Algorithm 1 and summarize ACC; (2) Randomly permute the class labels; (3) Repeat Algorithm 1, recording ACC^{PERM} at each run; (4) Repeat steps 2-3 Q times; and (5) Summarize comparison of ACC with ACC^{PERM} obtained by permuting the labels.

Significance in this case is a measure of whether or not the ACC achieved was better than chance, e.g. the permutation test. In the case of two groups with balance, i.e. the number of replicates in both groups equal, the null hypothesis with the permutation testing is defined as Ho: ACCTRUE=50% versus the alternative that Ha: ACCTRUE>50%. Hence, ACC arbitrarily close to 50% may be rejected as significant with enough samples, i.e. power, although ACC this low is rarely practical in medical decision making.

Results

Assessment of pathologic response. The overall pCR rate in the 82 patients was 26%, which is consistent with our previous experience in a larger randomized study using similar preoperative therapy (Green et al., 2001). Of the 8 factors listed in Table 2, only Age, Nuclear Grade, and ER status are significantly related to pCR when assessed individually. Preliminary analysis indicated that the probability of pCR was a parabolic function of age and this was confirmed with a univariate logistic regression model fitting age as a quadratic polynomial (p=0.0056). Estimated probabilities of complete response from this model are 10% for age 30, 38% for age 45, and 16% for age 60. The probability of pCR was 51% for ER negative patients, but only 6% for ER positive patients (p<0.0001). The probability of pCR was 38% for patients with Nuclear Grade 3, but only 6% for patients with lower grades (p=0.0006). In a logistic regression model with Age, Age2, Race=white, Tstage, Nstage>1, Nuclear-Grade>2, ER status, and HER2 status as predictors, only ER status (p=0.0037) and Age (p=0.012) were significant. The R-squared value was 38% and the area under the ROC curve was 90%.

Feature Selection To select informative genes for outcome prediction, expression data was compared in the highly chemotherapy sensitive (pCR) and more resistant tumors (cases with any residual disease). A beta uniform mixture (BUM) analysis of the p values showed a non-uniform distribution and was used to estimate false discovery rates (FDR) (Pounds and Morris, 2003). Setting the FDR to 5% resulted in 395 genes, 1% in 56 genes and 0.5% in 31 genes.

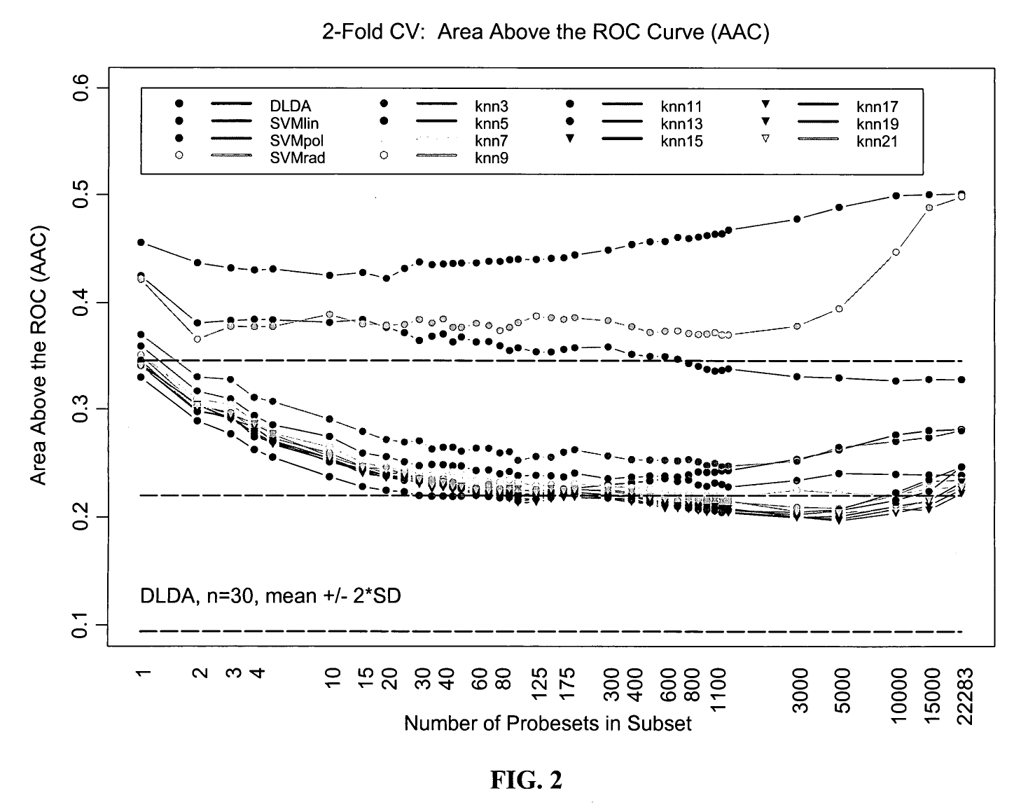

Development of multi-gene predictor of pathologic complete response. The inventors evaluated 14 classifier methods (SVM, DLDA, KNN k=3, 5, 7, 9, 11, 13, 15, 17, 19, 21) including various numbers of informative genes (39 values spanning the range 1 to 22,283, approximately equally spaced on the log scale) for a total of 546 classifiers. FIG. 2 shows the AAC results (means over the 100 iterations) for 2-fold CV plotting against the number of top genes included. The SVM classifiers clearly do worse than the others in this data set. The performance of the DLDA and KNN classifiers improves with increasing numbers of genes leveling off at about 80 genes. For classifiers with fewer than 80 genes, DLDA does slightly better achieving the best performance in this range at about 30 genes. A DLDA classifier with 30 genes has AAC about 22% with approximate 95% confidence intervals from 10% to 36%. Since the AAC results for most of the other classifiers (save for most of the SVM classifiers) fall within this confidence interval, these classifiers have performance that is statistically equivalent to those from DLDA with 30 genes. This indicates that there are many possible classifiers with very similar top performance.

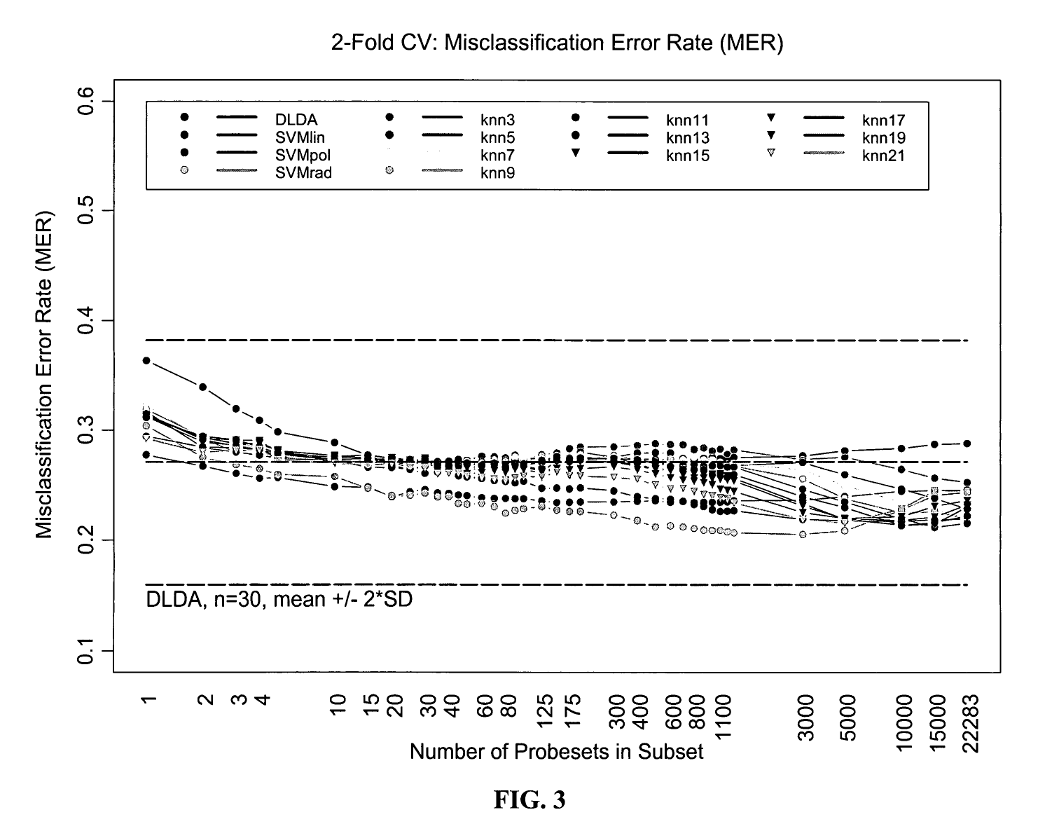

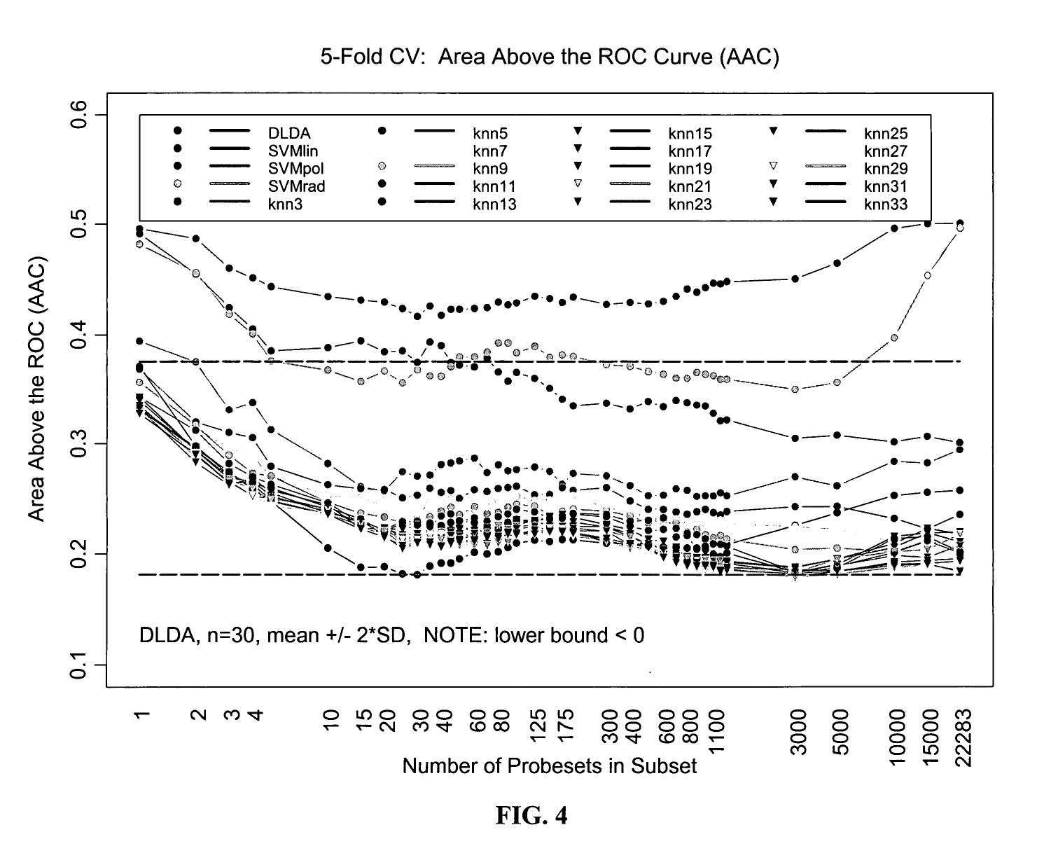

FIG. 3 is similar to FIG. 2 but showing MER instead of AAC. Here the results for all the classifiers are within a fairly tight envelop all falling within the 95% confidence interval for the results of DLDA with 30 genes (27%+/−12%). Two SVM classifiers actually have better performance than DLDA at 30 genes but by only about 5%, which is well within the margin of estimation error (SD=6%). FIG. 4 shows the results for AAC using 5-fold CV. The results are similar to the 2-fold CV, but with DLDA more clearly superior around 30 genes.

Intuitively, the inventors think a classifier with fewer genes than training samples makes sense to minimize overfitting and to yield a manageable number of genes. Also, the literature and inventor's experience suggest it can be problematic to rely on a small handful of genes. Somewhat arbitrarily, DLDA was selected using the 30 top genes as a single classifier to be tested on independent validation data. In addition to the MER and AAC results reported above, when using all 82 samples for training and testing, this classifier has 95% correct prediction among pCR patients, and 77% correct among RD patients. In addition, 59% of the patients predicted to be pCR were actually pCR, while 98% of the patients predicted to be RD actually were. After full 2-fold cross-validation, these values were: 65%, 75%, 47% and 87%, respectively.

To determine if this predictor performs significantly better than chance the inventors performed permutation testing in traditional 2-fold cross validation. The permutation test p-value was 0/1000, in other words none of the 1000 permuted data sets had accuracy as high or higher than that estimated from the original class labels. Permutation testing while allowing the genes to be resorted at each cross-validation iteration was deemed computationally prohibitive.

Prevalidation. A logistic regression model with the variables listed in Table 2 had an R-squared value of 38% and an area under the ROC curve of 90%. Adding the five top ranked genes to this model increased the R-squared value to 49%, the ROC area to 95% and yielded a likelihood ratio p-value for the new genes=0.0083. Since the inventors selected these genes as the most discriminatory from the array, this assessment is of course biased in favor of the genes.

To account for this, Tibshirani and Efron (2002) suggests using a pre-validation approach in which rather than including the expression value for the genes, the inventors include a cross-validated prediction from a multi-gene classifier (DLDA with 30 genes). The inventors used the proportion of pCR predictions from among the 100 repetitions of cross-validation as our value. Including this value in the model yielded a likelihood ratio p-value for the cross-validated predictions=0.0019 for standard cross-validation but p=0.75 for full cross-validation where the genes are resorted in each iteration.

The 30-gene DLDA itself yielded an ROC area of 92% when assessed on all 82 samples. This is comparable to the 90% ROC area for the logistic regression model based on the clinical variables. However, when the fully cross-validated values are used, the ROC area drops to 81%. There is no comparable value for the clinical data, since these variables are not being selected from a much larger set.

### Example 2

### Tau Expression as a Predictive Marker

Methods

Patients and specimens. This study was conducted at the Nellie B. Connally Breast Center of the University of Texas M. D. Anderson Cancer Center (MDACC). Sixty patients with newly diagnosed stage I-III breast cancer were included in the marker discovery study using gene expression profiling (LAB99-402). This prospective clinical study was approved by the institutional review board (IRB) and all patients signed an informed consent for voluntary participation. Fine-needle aspiration (FNA) was performed at the time of diagnosis before any treatment, and gene profiling was performed using Affymetrix U133A oligonucleotide probe arrays as previously reported (Symmans et al., 2003). All patients received 24 weeks of sequential T/FAC chemotherapy and underwent lumpectomy or modified radical mastectomy with axillary node sampling as determined appropriate by the surgeon. Complete pathologic response was defined as no histopathologic evidence of any residual invasive cancer cells in the breast and in the lymph nodes. The study population was described in detail previously (Ayers et al., 2004).

For immunohistochemical (IHC) validation a tissue microarray was used. The array was built from formaldehyde fixed, paraffin embedded tissues of pretreatment core needle biopsies from patients with stage I-III breast cancer. All patients received 24 weeks of preoperative chemotherapy with sequential paclitaxel and 5-fluorouracil, doxorubicin, cyclophosphamide on a clinical trial (MDACC DM 98-240) between December 1998 and April 2001 and subsequently underwent lumpectomy or modified radical mastectomy with axillary node sampling. One hundred and forty-three patients had pretreatment tissue available for tissue array analysis of Tau expression. Immunohistochemistry and data analysis were conducted in accordance with a laboratory protocol (LAB01-427) approved by the IRB of the University of Texas M. D. Anderson Cancer Center.

Twelve human breast tumor cell lines (T47D, BT20, ZR75.1, MCF7, MDA-MB-231, MDA-MB-361, MDA-MB 435, MDA-453, MDA-468, BT 549, BT 474 and SKBR3) were obtained from the American Type Culture Collection (ATCC, Manassas, Va.). All culture media components were purchased from the M. D. Anderson Tissue Culture Core Facility (Houston, Tex.).

Microarray data analysis. Microarray Suite 5.0 was used for data acquisition. dCHIP V1.3 (dchip.com) software was used for normalization across arrays. Probe set level intensity estimates were generated using the perfect match model (Stec et al., in press). To identify genes differentially expressed between cases with pathologic CR (n=18) and those with residual disease (n=42), probe sets were ordered by p-values obtained with two-sample t-tests with unequal variance on the ranks. A beta uniform mixture (BUM) analysis of the p values showed a non-uniform distribution and was used to estimate false discovery rates (Pounds and Morris, 2003). Setting the false discovery rate to 1% resulted in 19 probe sets, 4 out of the top 6 probe sets targeted the Tau gene.

Immunohistochemistry. Tissue microarrays were constructed with 0.6 mm diameter cores spaced 0.8 mm apart using a Tissue Microarray (Beecher Instruments, Inc). Two representative areas of each pre-chemotherapy core biopsy were selected for coring and placement in the tissue microarray. The tissue microarray blocks were cut to 5 μm sections. The tissue microarray slides were deparaffinized; and after blocking endogenous peroxidase activity and antigen retrieval (10 minutes high temperature microwave oven in citrate buffer, pH 6.0), the slides were incubated with anti-Tau antibody (1:50 dilution, clone T1029, US Biological) overnight at 4° C. Bound antibody was detected by using an antimouse horseradish peroxidase-labeled polymer secondary antibody (DAKO Envision TM+ System, DAKO, Carpentia, Calif.) then DAB substrate. Normal breast epithelium served as internal positive control and negative control included omission of the primary antibody. Cytoplasmic staining intensity was graded as either negative (0/1+) or positive (2+/3+). Slides were scored independently by 2 pathologists and without knowledge of the clinical outcome. Correlation with complete response was assessed in a univariate analysis (Chi square test) and a multivariate analysis including patient age, tumor size, histological type and grade, estrogen receptor, progesterone receptor and HER2 status and Tau staining intensity (logistic regression).

Small interfering RNA studies. Two siRNA oligonucleotides directed against microtubule associated protein Tau (genbank accession number NM_{—}016835.1) were ordered from Qiagen. Breast cancer cell lines were screened for Tau protein expression by Western blot analysis using a monoclonal anti-Tau antibody (#13-1400: clone T14, Zymed, CA). ZR75.1 cells were selected for siRNA studies and were transfected with a control siRNA (directed against lamin) or 2 distinct anti-Tau siRNA (5′-AATCACACCCAACGTGCAGAA-3′ (SEQ ID NO:194) and 5′-AACTGGCAGTTCTGGAGCAAA-3′) (SEQ ID NO:195) constructs. Five hundred nanograms of siRNA was transfected using 1.5 μl RNAiFect (Qiagen) onto 1-3×10⁴ cells in 96-well plates or 5 μg of siRNA was transfected using 15 μl RNAiFect (Qiagen) onto 1.5-4×10⁵ cells in 6-well plates following the manufactures instructions.

In vitro apoptosis and cell growth assays. Twenty-four hours after siRNA transfection, the medium was changed and cells were treated with various concentrations of paclitaxel and epirubicin. Proliferation rates were determined with CellTiter-Glo® Luminescent Cell Viability Assay, (Promega) after 48 hours of drug exposure according to the manufacturer's instructions. Chemosensitivity was determined from three separate experiments. Growth curves were generated with GraphPad Prism 4.01 (GraphPad Software, San Diego, Calif.). The effect of Tau expression on drug uptake was assayed using a fluorescent-conjugated paclitaxel (Oregon Green 488 paclitaxel, Molecular probes, Eugene, Oreg.) or spontaneously fluorescent epirubicin (Kimichi-Sarfaty et al., 2002; Harris et al., 2003). Forty-eight hours after siRNA transfection, 3×10⁵ cells were trypsinized and resuspended in 1 ml of regular medium containing 1 μM of fluorescent paclitaxel or 16 μM of epirubicin and incubated at 37° C. for 20 to 80 min. The pellet was resuspended in 400 μl of phosphate-buffered saline before FACS analysis (Kimichi-Sarfaty et al., 2002) using CellQuest software (BD Biosciences, San Jose, Calif.). Data were recorded by the FACScan as arbitrary units. The amount of fluorescence per cell (arbitrary fluorescence units) was taken as the measure of drug uptake. Results were displayed as histograms together with the mean fluorescence and standard deviation. The percentage of fluorescent cells versus non fluorescent cells was compared at least three times at 20, 50 and 80 minutes. Fluorescence paclitaxel uptake was also observed using an inverted fluorescent microscope.

Tubulin polymerization assays. Bovine brain tubulin (2 mg/ml) polymerization assays were performed in 100-μl volumes at 37° C. using the Tubulin Polymerization Assay Kit (Cytoskeleton, Inc., Denver, Colo.) and following the manufacturer recommendations. Purified Tau protein was purchased from Cytoskeleton (ref #TA01). Fluorescent Bodipy-paclitaxel was purchased from Molecular probes (Bodipy 564/570, Molecular probes, Eugene, Oreg.). OD340 was measured every 30 seconds for 30-60 min. The plots show the change in turbidity after correcting the data for the baseline absorbance.

Results

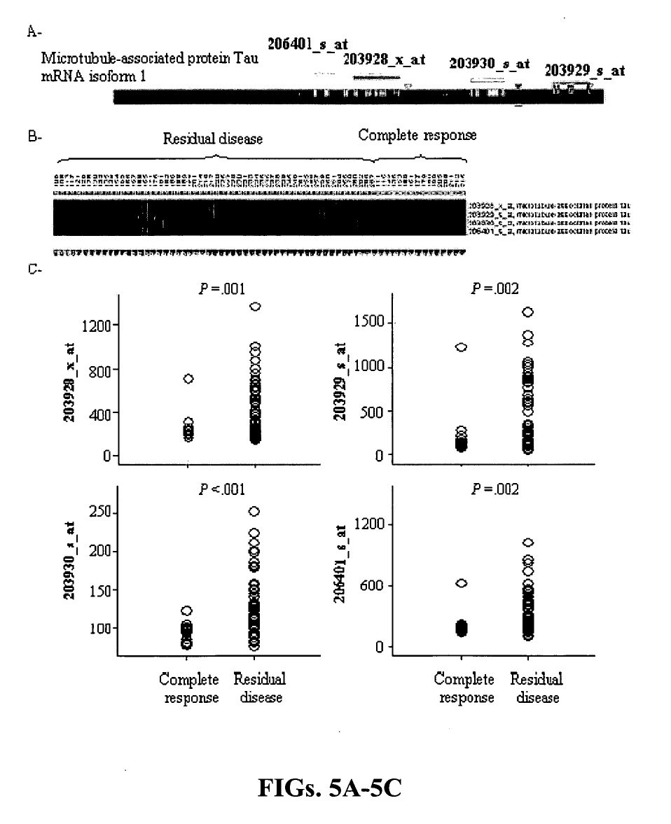

Low expression of Tau mRNA is associated with pathologic complete response to preoperative chemotherapy. To identify genes differentially expressed between cases with pathological CR (n=18) and those with residual cancer (n=42), all probe sets called present on the U133A chip were ordered by p-values obtained with two-sample t-tests with unequal variance on the ranks. The first (203930_s_at), third (203928_x_at), fourth (206401_x_at), and sixth probe sets (203929_s_at) on this list of differentially expressed genes all targeted the same gene, microtubule-associated protein Tau (NM_{—}16835.1). Tau mRNA expression was significantly lower (P<1.2×10⁻⁶) in tumors that achieved pathological CR. (FIG. 5). There was no differential expression of any of the other microtubule-associated proteins represented in our array data.

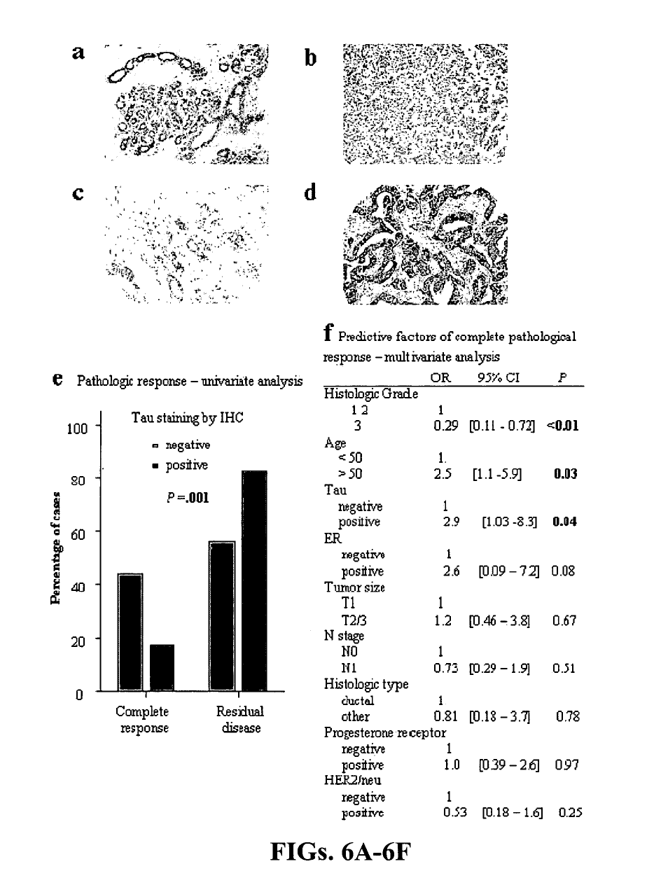

Validation of Tau expression with immunohistochemistry on tissue arrays in an independent patient population. Next, the inventors examined Tau protein expression in an independent set of cases using tissue microarrays of pre-chemotherapy core needle biopsies of breast cancer. The inventors performed immunohistochemistry (IHC) on 122 breast cancer tissues. All patients received 24 weeks of preoperative paclitaxel and anthracycline containing chemotherapy. None of these patients were included in the microarray study; therefore they represent an independent but identically treated validation group. Thirty-eight patients experienced pathological CR (31%). Cytoplasmic expression of Tau protein was seen in normal breast epithelium and blood vessels (FIG. 6A). Sixty-four tumors (52%) were considered Tau negative, including 14 with complete absence of Tau by immunohistochemistry (IHC score 0) and 50 tumors with less Tau expression than normal controls (IHC score 1+) (FIG. 6B). Fifty-eight tumors (48%) were positive for Tau protein expression, defined as IHC score 2+ that had uniform staining of similar or slightly greater intensity than normal contols (FIG. 6C) or IHC score 3+ that had uniform high intensity staining (FIG. 6D). This dichotomization of staining results was determined after inspection of the distribution of results and without knowledge of the clinical outcome data. There were more pathological CRs among the Tau-negative tumors ( 28/64, 44%) than among the Tau-positive tumors ( 10/58, 17%). Most tumors that achieved pathological CR were Tau-negative ( 28/38, 74%) (FIG. 6E). The odds ratio for pathological CR in Tau-negative tumors was 3.7 (95% confidence interval: 1.6-8.6, P=0.0013). A multiple logistic regression model with pathological CR as the outcome and age, tumor size, nodal status and histology, nuclear grade, estrogen receptor (ER), progesterone receptor (PR), and HER2 expression as covariates identified high nuclear grade (P<0.01), young age (P=0.03) and Tau-negative status (P=0.04) as independent predictive factors of pathological CR (FIG. 6F). A similar multiple logistic regression model with Tau as the outcome and including the same clinicopathological parameters as covariates identified low or intermediate nuclear grade (P=0.05), ER (P=0.06) and PR (P=0.005) as independent predictors of Tau status. ER-negative and high-grade tumors tended to be Tau-negative. The Tau-pCR odds ratio when adjusted for age, tumor size, nodal status and nuclear grade and ER, PR, and HER2 status was 2.7 (0.9, 7.9) with P=0.059. These results confirm the microarray data that low Tau expression is associated with higher probability of achieving pathological CR.

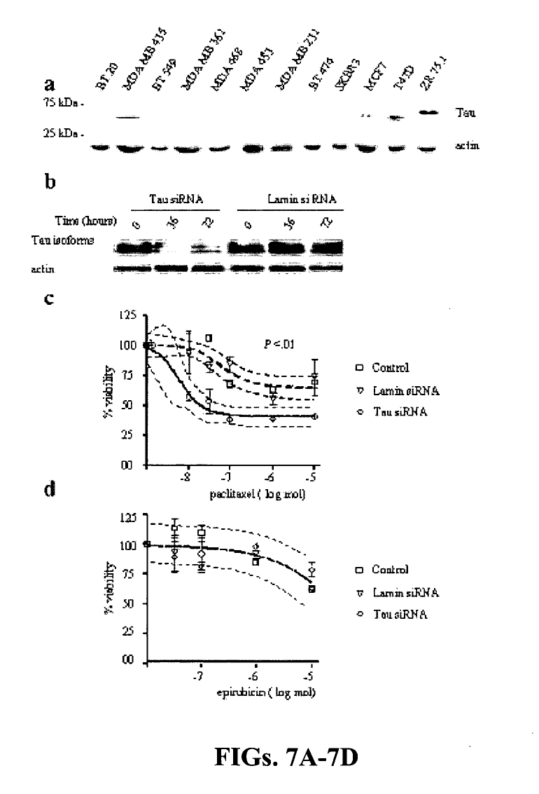

Down regulation of Tau expression in breast cancer cells increases sensitivity to paclitaxel in vitro. The inventors hypothesized that low Tau expression is not only a marker of response but contributes to increased sensitivity to paclitaxel chemotherapy due to its effect on microtubule assembly. The inventors assessed Tau protein expression in breast cancer cell lines with Western blot using an anti-Tau monoclonal antibody that recognizes Tau irrespectively of phosphorylation status. Four cell lines (ZR75.1, T47D, MCF7 and MDA-MB 435) expressed Tau, whereas eight other cell lines did not (FIG. 7A). ZR75.1 cells were selected for further in vitro studies because they express high levels of Tau protein and are known to be relatively resistant to paclitaxel (Dougherty et al., 2004). The invenotrs used siRNAs to reduce Tau protein expression and showed with the same antiboby used for the tissue array (clone T1029, US Biological, MA) that the nadir occurred 36 h after siRNA transfection (FIG. 7B). Twenty-four hours after siRNA transfection, cells were exposed to various concentrations of paclitaxel or epirubicin and cell viability was assessed after 48 h of drug exposure using an ATP cell viability assay. Decreased Tau expression by siRNA knock down significantly increased the sensitivity of ZR75.1 cells to paclitaxel compared to control cells transfected with lamin siRNA or no siRNA. (FIG. 7C). The IC₅₀ concentration of paclitaxel was reduced from >10 μM to 100 nM. Tau down-regulation did not result in increased sensitivity to epirubicin (FIG. 7D). These data demonstrate that Tau protein expression partially protects cells from the cytotoxic effects of paclitaxel. Induced suppression of Tau protein expression renders cells highly sensitive to this paclitaxel, but not epirubicin.

Tau protein reduces paclitaxel binding to tubulin and interferes with the paclitaxel induced stabilization in vitro. Tau is a microtubule-associated protein that promotes tubulin assembly and stabilizes polymerized tubulin. The inventor hypothesized that Tau may interfere with paclitaxel binding and pharmacological stabilization of tubulin. Intracellular paclitaxel is mostly bound to tubulin. To estimate paclitaxel binding to tubulin in the presence or absence of Tau protein, the uptake of fluorescent paclitaxel in Tau siRNA-treated (Tau knock down) cells and lamin siRNA-treated control cells were measured. Forty-four hours after siRNA transfection, cells were exposed to 1 μM Oregon green-paclitaxel for 20 to 80 min and then analyzed by FACS. The amount of fluorescent paclitaxel in the cells can be assessed by plotting fluorescence intensity in the X-axis and cell count on the Y-axis. Control ZR75.1 cells (lamin-siRNA) displayed a unimodal distribution (FIG. 8A) with low fluorescence intensity (mean: 4 units). In Tau-siRNA transfected ZR75.1 cells, the distribution of fluorescence intensity was bimodal with a fraction of highly fluorescent cells present (mean: 100 units) corresponding to the successfully transfected subpopulation of cells (FIG. 8B). When Tau expression was knocked down, the percentage of cells showing fluorescence over 10 units was 27.2% (+/−6.3) versus 7.2 (+/−0.8) in cells transfected with lamin siRNA (FIG. 8C). The same FACS experiment was conducted with epirubicin, which has spontaneous fluorescence. The distributions were unimodal and the fluorescence uptake was slightly decreased in the Tau knocked-down cells (FIGS. 8D and 16E). Using fluorescent microscopy, paclitaxel was visualized in the cytoplasm (FIG. 8F) and in the mitotic spindle (FIG. 8G) in Tau knocked-down cells. These data demonstrate that cells with lowered Tau protein expression accumulate more paclitaxel, but not epirubicin.

Microtubules are formed in vitro by non-covalent polymerization of tubulin dimers (Desai et al., 1997; Hong et al., 1998). Microtubule associated proteins, GTP and paclitaxel increase microtubule polymerization rates which can be measured by observing an increase in absorbance at 340 nm (Lu and Wood, 1993; Rao et al., 1999). The inventors hypothesized that Tau may reduce pharmacological tubulin polymerization induced by paclitaxel. The inventors performed a kinetic spectrophotometric tubulin polymerization assay in which Tau and paclitaxel were added together to the tubulin mixture. As shown in FIG. 9A, Tau and paclitaxel both induced tubulin polymerization and contrary to our expectation their combined effect was partially additive. Next, tubulin was pre-incubated with Tau before adding paclitaxel which approximates a more physiological sequence of drug exposure. Pre-incubation with Tau reduced the ability of paclitaxel to induce maximal tubulin polymerization in a dose-dependent manner (FIG. 9B). This phenomenon may have been due to reduced substrate availability because tubulin dimers already polymerized by Tau cannot be recruited by paclitaxel, or alternatively, Tau may directly compete with paclitaxel binding to tubulin.

To examine if paclitaxel binding to tubulin is affected by Tau expression, the inventors used fluorescent bodipy-paclitaxel. When fluorescent paclitaxel binds to microtubules it results in enhanced fluorescence. The inventors used this characteristic to assess the competition between Tau and paclitaxel in vitro. Fluorescent paclitaxel (5 μM) was added to a tubulin solution after 30 minutes pre-incubation with Tau (15 μM) or regular (non-fluorescent) paclitaxel (20 μM), or reaction buffer alone and fluorescence was measured 30 minutes later. Because of the insolubility of paclitaxel, the inventors were limited to 5 μM and had to make the samples with 25% bodipy-paclitaxel and 75% unlabelled paclitaxel to keep the DMSO concentration below 10%. As shown in FIG. 9C, the competition between fluorescent paclitaxel and unlabelled paclitaxel was very high and the fluorescence was low because fluorescent paclitaxel could not bind to the microtubules. In the control wells, the addition of fluorescent paclitaxel induced polymerization and after 30 minutes, fluorescence emission was high. When tubulin was pre-incubated with Tau there was less fluorescence, indicating that Tau partially inhibited paclitaxel binding to microtubules.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for assessing the responsiveness of a tumor to therapy comprising:
(a) obtaining a sample of a tumor from a cancer patient;
(b) evaluating the sample for expression of one or more markers identified in Table 1; and
(c) assessing the responsiveness of the tumor to therapy based on the evaluation of marker expression in the sample.

2. The method of claim 1 wherein the tumor is classified as sensitive, wherein the therapy achieves an outcome of a complete pathological response.

3. The method of claim 2, wherein the chance of a complete pathological response is at least 60%.

4. The method of claim 1, wherein the tumor is classified as unlikely to achieve complete pathological response to therapy.

5. The method of claim 4 wherein the chance of a complete pathological response is less than 15%.

6. The method of claim 1, wherein the therapy is P/FAC therapy.

7. The method of claim 1, wherein evaluating the expression of the one or more markers comprises using a prediction algorithm.

8. The method of claim 7, wherein the algorithm is k-nearest neighbor, support vector machines, diagonal linear discriminant analyses, or compound co-variate predictor.

9. The method of claim 8, wherein the algorithm is a k-nearest neighbor algorithm.

10. The method of claim 9, wherein the k-nearest neighbor algorithm is a k-nearest neighbor with a k=7.

11. The method of claim 1, wherein the tumor comprises breast cancer.

12. The method of claim 1, wherein the sample is obtained by aspiration, biopsy, or surgical resection.

13. The method of claim 1, wherein assessing the expression of the one or more markers comprises detecting mRNA of the one or more markers.

14. The method of claim 13, wherein the detection comprises microarray analysis.

15. The method of claim 14, wherein the microarray is further defined as an Affymetrix Gene Chip.

16. The method of claim 13, wherein the detection comprises PCR.

17. The method of claim 13, wherein the detection comprises in situ hybridization.

18. The method of claim 1, wherein assessing the expression of the one or more markers comprises detecting the protein encoded by one or more markers.

19. The method of claim 18, wherein detecting the protein is by immunohistochemistry.

20. The method of claim 1, wherein the marker is SEQ ID NO:1, microtubule-associated Tau.

21. The method of claim 20, wherein the therapy is P/FAC therapy.

22. The method of claim 20, wherein the tumor comprises breast cancer.

23. The method of claim 20, wherein the sample is obtained by aspiration, biopsy, or surgical resection.

24. The method of claim 20, wherein assessing the expression of SEQ ID NO:1 comprises detecting mRNA.

25. The method of claim 24, wherein the detection comprises PCR.

26. The method of claim 24, wherein the detection comprises in situ hybridization.

27. The method of claim 20, wherein assessing the expression of SEQ ID NO:1 comprises detecting a microtubule-associated Tau protein.

28. The method of claim 27, wherein detecting the protein is by immunohistochemistry.

29. A method of monitoring a cancer patient receiving P/FAC therapy comprising:
(a) obtaining a tumor sample from the patient during P/FAC therapy;
(b) evaluating expression of one or more markers of Table 1 in the tumor sample; and
(c) assessing the cancer patient's responsiveness to P/FAC therapy.

30. The method of claim 29, further comprising repeating steps (a) to (c) at various time points during P/FAC therapy.

31. The method of claim 29, wherein the marker is a microtubule-associated protein Tau marker.

32. A method of assessing anti-cancer activity of a candidate substance comprising:
(a) contacting a first cancer cell with the candidate substance;
(b) comparing expression of one or more markers in Table 1 in the first cancer cell with expression of the markers in a second cancer cell not contacted with the candidate substance; and
(c) assessing the anti-cancer activity of the candidate substance.

33. The method of claim 32, wherein the anti-cancer activity is sensitization of a cancer cell to therapy.

34. The method of claim 32, wherein the marker is a microtubule-associated protein Tau marker.

35. The method of claim 33, wherein the therapy is a chemotherapy.

36. The method of claim 35, wherein the chemotherapy is P/FAC therapy.
